SEQUENCE LISTING (77 sequences)

SEQ ID NO: 1 - protein, 523 aa, Saccharomyces cerevisiae
MGEDFMHPPFQTYPSKNSEGKKHIVIVGGGIIGCCTAYYLTQHPSFSPSTHHITIIESRRIAGGASGKAGGLLASWAFPHQIVPLSFQLHQELSDEYDGENNWDYRRLTTVSLEADVREEVIENYERLSKKAYNLNVPPPKKRPGYISNKFNIGDSNSSLSSSGSSLKNDSASNEEEGSDIHVSSSVPSLHSLTNERMRSHTNSASDLDSVSPVEQLRETNIHNPLPADLDWIRRELVNDWSSLGGTDTTAQLHPYKFTHFILSKAMETGAVDLLLGKVVGLKCDEMDCVHSLKYLPSVVKNRRNSRGHAENPDIKLGTIFNDENAKPIEINDIQQIVLSMGPWTSKILKDCPISGLRAHSVTIKPSEKTVSPYAILAELKVNDREFFSPEMYARKDEVYVCGEGDTLVNIPESSDDVEVVSEKCDELYHYVSKLSPTLSKGHLLRKQACFLPVLNVPTSSGPLIGETNVKDLYIASGHSCWGINNAPATGKLMAEILLDGEATSAEISSLDPKLYFDATILS

SEQ ID NO: 2 - protein, 223 aa, Saccharomyces cerevisiae
MSSQPSFVTIRGKAISLETQTESLLSKYSTFAQTTSSEQTGQEKKIDKQLEGILGQRQDVIDSLTQICDSNPAISASKLSQLHRHKEILQDHWKSFRNIRSSIQQERNRLNLLFSVKNDIANSTTDAPAPIGDADEYIQNETRRIDQSNNVVDRLISQAWETRSQFHSQSNVLNTANNKVLQTLQRIPGVNQLIMKINTRRKKNAFVLATITTLCILFLFFTW

SEQ ID NO: 3 - protein, 675 aa, Saccharomyces cerevisiae
MDYEDNLEAPVWDELNHEGDKTQSLVSNSIESIGQISTNEERKDNELLETTASFADKIDLDSAPEWKDPGLSVAGNPQLEEHDNSKADDLINSLAPEQDPIADLKNSTTQFIATRESGGALFTGNANSPLVFDDTIYDANTSPNTSKSISGRRSGKPRILFDSARAQRNSKRNHSLKAKRTTASDDTIKTPFTDPLKKAEKENEFVEEPLDDRNERRENNEGKFTASVEKNILEQVDRPLYNLPQTGANISSPAEVEENSEKFGKTKIGSKVPPTEKAVAFKVEVKDPVKVGELTSIHVEYTVISESSLLELKYAQVSRRYRDFRWLYRQLQNNHWGKVIPPPPEKQSVGSFKENFIENRRFQMESMLKKICQDPVLQKDKDFLLFLTSDDFSSESKKRAFLTGSGAINDSNDLSEVRISEIQLLGAEDAAEVLKNGGIDAESHKGFMSISFSSLPKYNEADEFFIEKKQKIDELEDNLKKLSKSLEMVDTSRNTLAASTEEFSSMVETLASLNVSEPNSELLNNFADVHKSIKSSLERSSLQETLTMGVMLDDYIRSLASVKAIFNQRSKLGYFLVVIENDMNKKHSQLGKLGQNIHSEKFREMRKEFQTLERRYNLTKKQWQAVGDKIKDEFQGFSTDKIREFRNGMEISLEAAIESQKECIELWETFYQTNL

SEQ ID NO: 4 - protein, 551 aa, Saccharomyces cerevisiae
MTSAVPYDPYDDLDNNPFAEPQEEDSEPAATTTDGSSSMSEERVGTEQTAASVQDNGTANNIQNGLGEEGNATRSKTSNEHNENQQPSQPSERVILPERSDEKKKYTLLAKVTGLERFGSATGKKENPTIIFDCSTNLPTFRKQQYKNVKKSYEEFHQLFKYLNVAIQESFVPTLPSAYTTFGINSEEDRMKVTRNFQLWFNRLSQDPLIIRNEEVAFFIESDFNTYTPINKSKSLASGLKRKTLKQLAPPYDEITELAEFRPLVKSIYVVSQSLQEKLLRVSRNRKMMVQEENAFGQDFVNLDEHNKLYRRYGKILTAVGDIDSIIATMDMATLYDGLEWIVRDAYAVKEALTNRHFIMRNLVQAQQNSKAKQEQARRFRSRRDINPMKIDEALRQLKAAAKNEQVLTLKLQRITSNMIIERKQWISWYEEWIRSSIKEFTLRKIEYERKKLTLLERVRSDIRKADENGGLSRLGRHAVSNNNSDTSQTLKGDSWTGESNRKSQIPINKIAHTEFDDELFTEDDGYNSQDSDTTSLNARHAASLLGMSTK

SEQ ID NO: 5 - protein, 379 aa, Saccharomyces cerevisiae
MSIFFKPPIDIEILFDNEESRKHVDIATRSSNSSYKSMKESLPVYEDGESLGGIVTLRVRDSKKVDHLGIKVSVIGSIDMLKSHGSGNSSSKKVTSSTSSSSSNGSVDVRKNSVDQFLCQSYDLCPAGELQHSQSFPFLFRDLSKRYESYKGKNVDVAYYVKVTVMRKSTDISKIKRFWVYLYNSVTTAPNTLSANETKATTNDIAGGNYAADNASDNTQTKSTQGEAADVNQVLPISHSNNEPKPVRLDIGIENCLHIEFEYAKSQYSLKEVIVGRIYFLLTRLRIKHMELSLITRESSGLQTSNVMTDSTAIRYEIMDGSSVKGETIPIRLFLSGYDLTPNMSCNYFNVKNYLSLVIIDEDGRRYFKQSEITLYRTR

SEQ ID NO: 6 - protein, 282 aa, Saccharomyces cerevisiae
MLLLALSDAHIPDRATDLPVKFKKLLSVPDKISQVALLGNSTKSYDFLKFVNQISNNITIVRGEFDNGHLPSTKKDKASDNSRPMEEIPMNSIIRQGALKIGCCSGYTVVPKNDPLSLLALARQLDVDILLWGGTHNVEAYTLEGKFFVNPGSCTGAFNTDWPIVFDVEDSDEAVTSEVDKPTKENQSEDDDAKGGSTGKEQPGSYTPKEGTAGERENENESNVKPENQFKEDEVDMSDSDINGSNSPSFCLLDIQGNTCTLYIYLYVNGEVKVDKVVYEKE

SEQ ID NO: 7 - protein, 944 aa, Saccharomyces cerevisiae
MAYADSPENAIAVIKQRTALMNRCLSQHKLMESLQHTSIMLTELRNPNLSPKKYYELYVIIFDSLTNLSTYLIENHPQNHHLADLYELVQYTGNVVPRLYLMITVGTSYLTFNEAPKKEILKDMIEMCRGVQNPIRGLFLRYYLSQRTKELLPEDDPSFNSQFIMNNFIEMNKLWVRLQHQGPLRERETRTRERKELQILVGSQLVRLSQIIDDNFQMYKQDILPTILEQVIQCRDLVSQEYLLDVICQVFADEFHLKTLDTLLQTTLHLNPDVSINKIVLTLVDRLNDYVTRQLEDDPNATSTNAYLDMDVFGTFWDYLTVLNHERPDLSLQQFIPLVESVIVLSLKWYPNNFDNLNKLFELVLQKTKDYGQKNISLESEHLFLVLLSFQNSKLQLTSSTTAPPNSPVTSKKHFIFQLISQCQAYKNILALQSISLQKKVVNEIIDILMDREVEEMADNDSESKLHPPGHSAYLVIEDKLQVQRLLSICEPLIISRSGPPANVASSDTNVDEVFFNRHDEEESWILDPIQEKLAHLIHWIMNTTSRKQTMKNKIQFSLEAQLEILLLIKSSFIKGGINVKYTFPAIITNFWKLMRKCRMIQEYLLKKRPDNKTLLSHYSNLLKQMFKFVSRCINDIFNSCNNSCTDLILKLNLQCAILADQLQLNEISYDFFSQAFTIFEESLSDSKTQLQALIYIAQSLQKTRSLYKEAYYDSLIVRCTLHGSKLLKKQDQCRAVYLCSHLWWATEISNIGEEEGITDNFYRDGKRVLECLQRSLRVADSIMDNEQSCELMVEILNRCLYYFIHGDESETHISIKYINGLIELIKTNLKSLKLEDNSASMITNSISDLHITGENNVKASSNADDGSVITDKESNVAIGSDGTYIQLNTLNGSSTLIRGVVATASGSKLLHQLKYIPIHHFRRTCEYIESQREVDDRFKVIYV

SEQ ID NO: 8 - protein, 674 aa, Saccharomyces cerevisiae
MSRKNSKKLKVYYLPVTLTQFQKDLSEILISLHAKSFKASLIGEPQADAVNKPSGLPAGPETHPYPTLSQRQLTYIFDSNIRAIANHPSLLVDHYMPRQLLRMEPTESSIAGSHKFQVLNQLINSICFRDREGSPNEVIKCAIIAHSIKELDLLEGLILGKKFRTKRLSGTSLYNEKHKFPNLPTVDSTINKDGTPNSVSSTSSNSNSTSYTGYSKDDYDYSVKRNLKKRKINTDDWLFLATTKHLKHDQYLLANYDIDMIISFDPMLEIELPALQVLRNNANKDIPIIKLLVQNSPDHYLLDSEIKNSSVKSSHLSNNGHVDDSQEYEEIKSSLLYFLQARNAPVNNCEIDYIKLVKCCLEGKDCNNILPVLDLITLDEASKDSSDSGFWQPQLTKLQYSSTELPLWDGPLDIKTYQTDLMHRAVIRLRDIQDEYAKGTVPLYEKRLNETQRQNQLDEIKNSVGLTFKKKQEMEKSINDSEKRLKHAMTESTKLQNKINHLLKIRQELENFNKLPSNTTSSENHLEEGSALADKLKEYIDKNATLFNKLKELQQANAEKSKLNDELRSKYQIESSKAAESAQTLKILRESMKSLENEVNGPLTKFSTESLKKELERLQNDFQSLKARNKFLKNYITLMNRQYDLKNKNNVQVEKAAANGTRFRSTRSNTPNYT

SEQ ID NO: 9 - protein, 655 aa, Saccharomyces cerevisiae
MDLLRILDTKPIPTIVDATTLGISGNTSGDYWLPTTMSLYQKELTDQIVSLHYSDILRYFETSHYKEDVILESMKTMCLNGSLVATHPYLLIDHYMPKSLITRDVPAHLAENSGKFSVLRDLINLVQEYETETAIVCRPGRTMDLLEALLLGNKVHIKRYDGHSIKSKQKANDFSCTVHLFSSEGINFTKYPIKSKARFDMLICLDTTVDTSQKDIQYLLQYKRERKGLERYAPIVRLVAINSIDHCTLFFGKKFDKNSREYLENVTAAMVILRDRLGTLPPDLRPIYSQKLHYLVEWLENPTVPWPLPDIYPLKQYTSMDVERSLLTEVHFKKSDDQLEDAFSNCSKKRGRHGANKAASSTVAGIEDNITPSFYSTKRLKNDYYTNPLKQDMTQLTGITTADNSSNVNYHLSSGIITHKLIQSMGEVYMDICVQKQELDDYSCLDDLQNDHLKFFSNEDEKIIKEYETVLRTNNDNLNRSHELEVENNLKFSQIETLEKDIETLKGSLMAQGETLSKLKDAFVKTDNVQDEIEKEERISVSRDTEKKYMEQEIKRAVDAIRENEEETHKLNEKKNGLESELKLKFGKSEISTKELNEKIGFLKKELKLENDLNEELMGQLSKTMDNLENLTIPRVRTQNGNTKKKSKAKKPGNV

SEQ ID NO: 10 - protein, 569 aa, Saccharomyces cerevisiae
MSFQIETVPTKPYEDQKPGTSGLRKKTKVFKDEPNYTENFIQSIMEAIPEGSKGATLVVGGDGRYYNDVILHKIAAIGAANGIKKLVIGQHGLLSTPAASHIMRTYEEKCTGGIILTASHNPGGPENDMGIKYNLSNGGPAPESVTNAIWEISKKLTSYKIIKDFPELDLGTIGKNKKYGPLLVDIIDITKDYVTFLKEIFDFDLIKKFIDNQRSTKNWKLLFDSMNGVTGPYGKAIFVDEFGLPADEVLQNWHPSPDFGGMHPDPNLTYASSLVKRVDREKIEFGAASDGDGDRNMIYGYGPSFVSPGDSVAIIAEYAAEIPYFAKQGIYGLARSFPTSGAIDRVAKAHGLNCYEVPTGWKFFCALFDAKKLSICGEESFGTGSNHVREKDGVWAIMAWLNILAIYNKHHPENEASIKTIQNEFWAKYGRTFFTRYDFEKVETEKANKIVDQLRAYVTKSGVVNSAFPADESLKVTDCGDFSYTDLDGSVSDHQGLYVKLSNGARFVLRLSGTGSSGATIRLYIEKYCDDKSQYQKTAEEYLKPIINSVIKFLNFKQVLGTEEPTVRT

SEQ ID NO: 11 - protein, 870 aa, Saccharomyces cerevisiae
MSTTLAAPAKLKSLLLNLHTHCIGLHVNDVTPKVYFKLLIRHLLQISRSNAAHPKLRRRAQILLVSLFLSGVTLFSGVTYSTFKIILKCYKFYKFPWKRRNRRPLIRRTRSQMQLDSGARIMYIPEVELVDRQSPDDNKFMNATDKKKRKRIFIPPKDNDVYEHDKFLFKNVELERAKNSQLFYSKFLNQMNVLSKILIPTVFDKNFLLLTAQIFFLVMRTWLSLFVAKLDGQIVKNIIAGRGRSFLWDLGCWFLIAVPASYTNSAIKLLQRKLSLNFRVNLTRYIHDMYLDKRLTFYKLIFDAKASNSVIKNIDNSITNDVAKFCDATCSVFANIAKPVIDLIFFSVYLRDNLGTVGVAGIFVNYFITGFILRKYTPPLGKLAGERSASDGDYYNYHLNMINNSEEIAFYQGTAVERTKVKELYDVLMEKMLLVDKVKFGYNMLEDYVLKYTWSGLGYVFASIPIVMSTLATGINSEEKNMKEFIVNKRLMLSLADAGSRLMHSIKDISQLTGYTNRIFTLLSVLHRVHSLNFNYGAVPSILSIRTEDASRNSNLLPTTDNSQDAIRGTIQRNFNGIRLENIDVIIPSVRASEGIKLINKLTFQIPLHIDPITSKSNSIQDLSKANDIKLPFLQGSGSSLLILGPNGCGKSSIQRIIAEIWPVYNKNGLLSIPSENNIFFIPQKPYFSRGGTLRDQIIYPMSSDEFFDRGFRDKELVQILVEVKLDYLLKRGVGLTYLDAIADWKDLLSGGEKQRVNFARIMFHKPLYVVLDEATNAISVDMEDYLFNLLKRYRFNFISISQRPTLIKYHEMLLEIGENRDGKWQLQAVGTDEAITSIDNEIEELERKLERVKGWEDERTKLREKLEII

SEQ ID NO: 12 - protein, 853 aa, Saccharomyces cerevisiae
MISTASAFYQKHRVNLLRSSYIILLLATLYNSNSSSSNNKTDKKDSESTVLENKKIEEGKETAVDREEDESSKEELTIVSKHSTDSEDGAIIIDKESKTNHKGGERKGKVDFLFKLLLHDKKCLILFITQAILLNIRTLLSLRVATLDGQLVSTLVRAQYANFTKILLGKWMILGIPASFINSLISYTTKLCAVTINRKVSDFLLSKYLSNHHTFYSVASAESVSEIQDNLTKDIYTFSMNSSLLLNQLLKPMLDLILCSFKLLTSNTSVMGEGTLALGLIVYASNSLLKLIQPNFTRLTMASASLESWFRSLHSNLHSSNEEIALLRGQKRELENVDYSFYRLVLFLNREIKARAIYDVATAFVIKYTWGAAGLVLCSIPIFFKNKPSEDTLQLKEPGNDMTADFITNRRLLVTASSSIGRFVELKRNIQQLRGIRLRLNKFNDLLDANKGDDEKEPRDERCIVEYDDSRIKFENIPLITPANQVLVPELSFDLKHGNHLLIIGPNGCGKSSLFRILGGLWPIRATPNKNHQSKLIMPRRTVDRDCAIFYLPQRPYMGNRSTFREQIIYPDSIEQFKERYHNDYDLGDADLIKILQLLDLEDLVTENMSLLLAQRTSKNDSQQLSTEDNQSPCAIKVRDAFSIVRNWSEELTIGVQQRLAMARMYYHKPKFAVLDECTSAVAPEMEQRMYENAQNFGISLISVCHRTSLWHFHNYLLKFDGKGGYQFGPFNPKERLCNEEKLLELNAILDQQVPLWERKLKDLTIAKESNIIRKSETNLNLFEKIEDPKTSKSNALFNANKGQRITSPTGQETSKRLPLFSQPSSSASSNLLRNNKSLNKKVKTKKEEGKER

SEQ ID NO: 13 - protein, 760 aa, Saccharomyces cerevisiae
MKITCTDLVYVFILLFLNTSCVQAVFSDDAFITDWQLANLGPWEKVIPDSRDRNRVLILSNPTETSCLVSSFNVSSGQILFRNVLPFTIDEIQLDSNDHNAMVCVNSSSNHWQKFDLHDWFLLEEGVDNAPSTTILPQSSYLNDQVSIKNNELHILDEQSKLAEWKLELPQGFNKVEYFHREDPLALVLNVNDTQYMGFSANGTELIPVWQRDEWLTNVVDYAVLDVFDSRDVELNKDMKAELDSNSLWNAYWLRLTTNWNRLINLLKENQFSPGRVFTKLLALDAKDTTVSDLKFGFAKILIVLTHDGFIGGLDMVNKGQLIWKLDLEIDQGVKMFWTDKNHDELVVFSHDGHYLTIEVTKDQPIIKSRSPLSERKTVDSVIRLNEHDHQYLIKFEDKDHLLFKLNPGKNTDVPIVANNHSSSHIFVTEHDTNGIYGYIIENDTVKQTWKKAVNSKEKMVAYSKRETTNLNTLGITLGDKSVLYKYLYPNLAAYLIANEEHHTITFNLIDTITGEILITQEHKDSPDFRFPMDIVFGEYWVVYSYFSSEPVPEQKLVVVELYESLTPDERLSNSSDNFSYDPLTGHINKPQFQTKQFIFPEIIKTMSISKTTDDITTKAIVMELENGQITYIPKLLLNARGKPAEEMAKDKKKEFMATPYTPVIPINDNFIITHFRNLLPGSDSQLISIPTNLESTSIICDLGLDVFCTRITPSGQFDLMSPTFEKGKLLITIFVLLVITYFIRPSVSNKKLKSQWLIK

SEQ ID NO: 14 - protein, 117 aa, Saccharomyces cerevisiae
MSSSTPFDPYALSEHDEERPQNVQSKSRTAELQAEIDDTVGIMRDNINKVAERGERLTSIEDKADNLAVSAQGFKRGANRVRKAMWYKDLKMKMCLALVIIILLVVIIVPIAVHFSR

SEQ ID NO: 15 - protein, 115 aa, Saccharomyces cerevisiae
MSSSVPYDPYVPPEESNSGANPNSQNKTAALRQEIDDTVGIMRDNINKVAERGERLTSIEDKADNLAISAQGFKRGANRVRKQMWWKDLKMRMCLFLVVIILLVVIIVPIVVHFS

SEQ ID NO: 16 - protein, 403 aa, Saccharomyces cerevisiae
MTIAPMANDLEDFESLLEPTFDAKQFGNDLLKATNNNDTTILDLNTPLKKLNYDLHEIDSRIDQLMNSNPLEIIELIYKNEHVNSTIVGELKPSLEYMNMSYDRLKNQVLDPYERARKVQLALSKVYQTSFLLRGALLYIHLSNKLNALSKTAQLSTSTAINLASLHYQLEITLDENKNLKSLRKIKQLDQDIVSPNKRELITFLSLQMCKECLNSIKIKSNKEIISQLAYSLYLLSSQEFESAINKIVLSNVTMSSQILSKILNSIRMFPDAFNEVVEKGYNIYILETLLRNIKTDNVTNSSRSIAANKSRLGNLLSEYTSMKSKAGSGTPRDLFWSKVSSAFKKDFEISVNRGGPVGKSLLKNKDFIINTMKQSMKKSSDNSDYQSYLDVMLNSVSISLNK

SEQ ID NO: 17 - DNA, 1569 bp, Saccharomyces cerevisiae
  source 1..1569: mol_type = genomic DNA
atgaagttttctgctggtgccgtcctgtcatggtcctccctgctgctcgcctcctctgttttcgcccaacaagaggctgtggcccctgaagactccgctgtcgttaagttggccaccgactccttcaatgagtacattcagtcgcacgacttggtgcttgcggagttttttgctccatggtgtggccactgtaagaacatggctcctgaatacgttaaagccgccgagactttagttgagaaaaacattaccttggcccagatcgactgtactgaaaaccaggatctgtgtatggaacacaacattccagggttcccaagcttgaagattttcaaaaacagcgatgttaacaactcgatcgattacgagggacctagaactgccgaggccattgtccaattcatgatcaagcaaagccaaccggctgtcgccgttgttgctgatctaccagcttaccttgctaacgagacttttgtcactccagttatcgtccaatccggtaagattgacgccgacttcaacgccaccttttactccatggccaacaaacacttcaacgactacgactttgtctccgctgaaaacgcagacgatgatttcaagctttctatttacttgccctccgccatggacgagcctgtagtatacaacggtaagaaagccgatatcgctgacgctgatgtttttgaaaaatggttgcaagtggaagccttgccctactttggtgaaatcgacggttccgttttcgcccaatacgtcgaaagcggtttgcctttgggttacttattctacaatgacgaggaagaattggaagaatacaagcctctctttaccgagttggccaaaaagaacagaggtctaatgaactttgttagcatcgatgccagaaaattcggcagacacgccggcaacttgaacatgaaggaacaattccctctatttgccatccacgacatgactgaagacttgaagtacggtttgcctcaactctctgaagaggcgtttgacgaattgagcgacaagatcgtgttggagtctaaggctattgaatctttggttaaggacttcttgaaaggtgatgcctccccaatcgtgaagtcccaagagatcttcgagaaccaagattcctctgtcttccaattggtcggtaagaaccatgacgaaatcgtcaacgacccaaagaaggacgttcttgttttgtactatgccccatggtgtggtcactgtaagagattggccccaacttaccaagaactagctgatacctacgccaacgccacatccgacgttttgattgctaaactagaccacactgaaaacgatgtcagaggcgtcgtaattgaaggttacccaacaatcgtcttatacccaggtggtaagaagtccgaatctgttgtgtaccaaggttcaagatccttggactctttattcgacttcatcaaggaaaacggtcacttcgacgtcgacggtaaggccttgtacgaagaagcccaggaaaaagctgctgaggaagccgatgctgacgctgaattggctgacgaagaagatgccattcacgatgaattgtaa

SEQ ID NO: 18 - DNA, 2502 bp, Saccharomyces cerevisiae
  source 1..2502: mol_type = genomic DNA
atgaataatgctgcaaatacagggacgaccaatgagtcaaacgtgagcgatgctccccgtattgagcctttaccaagcttgaatgatgatgacattgaaaaaatcttacaaccgaacgatatctttacgaccgatcgtaccgatgcaagtactacatcttccacagccattgaagatattattaacccctcattggatccgcagtcagcagcatcgccggttccttcttcctcttttttccatgactcaaggaaaccttccaccagtacacatttagtaaggagaggtactccattgggaatttaccaaaccaatctatacggtcacaatagcagagaaaatactaatcctaatagtacattattatcttctaagttactcgcgcatccaccagttccttatgggcaaaatcccgatttactacaacatgctgtgtacagggcacagccgtcaagtggaaccactaacgcgcaaccgcgccaaaccacaagaagatatcaatcccataaatcacggcctgcatttgttaataaactatggagcatgttaaacgatgattctaatacgaaacttatacagtgggcggaggatggaaaatcttttattgtcacgaatagggaggaatttgtgcaccaaattttaccaaaatattttaaacattccaatttcgcttcctttgtaagacaattgaacatgtatggatggcataaagttcaagatgtcaagtcaggatcaattcaaagtagttcagatgataagtggcaatttgaaaatgaaaacttcattagaggtagagaagatttgctggaaaaaataatcaggcagaaaggttcctccaataaccataatagccctagtggtaacggtaatccagcgaatggtagcaacatccctctggacaatgccgcaggaagtaataatagcaataataacatcagtagtagtaattcattttttaacaatggtcatttattgcagggtaaaacactaagattaatgaacgaagcgaatcttggagataagaatgatgtcaccgcgattttgggggaattagagcaaataaaatataaccagattgcaatttccaaagatttactaagaataaacaaagataatgagttattatggcaagagaatatgatggccagggaaagacatagaacccaacagcaagccttggaaaaaatgttcagattcttgacatctatagtcccacacttagatcccaaaatgattatggacgggctgggagatccgaaagttaataatgaaaagctaaacagtgcgaataacattgggttaaatcgcgacaacacaggcactatagatgaactaaaatccaacgattctttcataaacgatgatcgtaattctttcaccaatgctacaaccaacgcccgtaataacatgagtcccaacaatgatgacaatagtattgacaccgctagcactaataccaccaacagaaagaaaaatatagatgaaaacatcaaaaataacaacgacataattaatgacattatatttaataccaaccttgccaacaatctcagcaattacaattccaacaataatgctggctcgccaataaggccctataaacaaagatatcttttgaaaaatagagccaattcctcgacatcgagtgagaatccaagcctaacgccctttgatatcgaatctaataatgaccgcaaaatttcagaaattccttttgatgacgaagaagaagaagaaacggattttaggccttttacctcgcgagatcctaataaccaaacgagtgaaaacacttttgatccaaacagatttacgatgctctctgatgatgatttaaaaaaagattctcataccaatgacaataaacacaacgaaagtgatcttttttgggacaacgtacatagaaatatagacgaacaagatgcaagactccagaacttggaaaatatggttcacatactttctcctggatatcctaataagtcgttcaacaacaaaacttcctcgacaaacactaattccaatatggaaagtgctgtcaacgttaatagccctggtttcaacttacaggattatttaactggagagtctaattcccccaattctgttcattctgttccctccaatggcagcggctccacaccgttgcccatgccaaatgataatgacaccgagcacgcaagtacaagtgtcaatcaaggcgaaaatggaagcggattaacgcccttcctcacggtagatgatcacacactaaacgacaataacactagtgagggaagtacaagggtgtcccccgatataaagttcagcgccactgaaaacactaaagtgagtgataacctgccaagctttaatgaccacagttattccacccaggccgacacggcgcccgagaacgctaagaaaagatttgtggaggaaataccggaaccggctatagtcgaaatacaggacccgacagagtacaacgatcaccgcctgcccaaacgagctaagaaatag

SEQ ID NO: 19 - protein, 238 aa, Saccharomyces cerevisiae
MEMTDFELTSNSQSNLAIPTNFKSTLPPRKRAKTKEEKEQRRIERILRNRRAAHQSREKKRLHLQYLERKCSLLENLLNSVNLEKLADHEDALTCSHDAFVASLDEYRDFQSTRGASLDTRASSHSSSDTFTPSPLNCTMEPATLSPKSMRDSASDQETSWELQMFKTENVPESTTLPAVDNNNLFDAVASPLADPLCDDIAGNSLPFDNSIDLDNWRNPEAQSGLNSFELNDFFITS

SEQ ID NO: 20 - DNA, 41 bp, synthetic construct
  misc_feature 1..41: note = Primer
  source 1..41: mol_type = other DNA
actgcggccgcaacaaaatgtcttacagaggacctattgga

SEQ ID NO: 21 - DNA, 40 bp, synthetic construct
  misc_feature 1..40: note = Primer
  source 1..40: mol_type = other DNA
cgtgagctcctagtaaatcttcttcttttcatcaacggat

SEQ ID NO: 22 - DNA, 37 bp, synthetic construct
  misc_feature 1..37: note = Primer
  source 1..37: mol_type = other DNA
tcaggatccaacaaaatgacaatagcgccaatggcaa

SEQ ID NO: 23 - DNA, 45 bp, synthetic construct
  misc_feature 1..45: note = Primer
  source 1..45: mol_type = other DNA
ccaggtacctcacttatttagagaaatagatactgagtttagcat

SEQ ID NO: 24 - DNA, 38 bp, synthetic construct
  misc_feature 1..38: note = Primer
  source 1..38: mol_type = other DNA
actgcggccgcaacaaaatgaagttttctgctggtgcc

SEQ ID NO: 25 - DNA, 37 bp, synthetic construct
  misc_feature 1..37: note = Primer
  source 1..37: mol_type = other DNA
cgtgagctcttacaattcatcgtgaatggcatcttct

SEQ ID NO: 26 - DNA, 80 bp, synthetic construct
  misc_feature 1..80: note = Primer
  source 1..80: mol_type = other DNA
atgagtaggaaaaattctaagaaactaaaagtctattacttacctgtaacgctaacccaagacatggaggcccagaatac

SEQ ID NO: 27 - DNA, 113 bp, synthetic construct
  misc_feature 1..113: note = Primer
  source 1..113: mol_type = other DNA
aaatctctctatattatacaggctacttcttttaggaaacgtcacattcattagtcgatagtattgtatctattttctttatttttcacacaccagtatagcgaccagcattc

SEQ ID NO: 28 - DNA, 83 bp, synthetic construct
  misc_feature 1..83: note = Primer
  source 1..83: mol_type = other DNA
atggactacgaggataatctagaagcacctgtttgggacgaactaaatcatgagggagataaagacatggaggcccagaatac

SEQ ID NO: 29 - DNA, 113 bp, synthetic construct
  misc_feature 1..113: note = Primer
  source 1..113: mol_type = other DNA
ataaatcctgaggaacgtgacacataaagttattgtatacagatcatctattaggcttgttattgcaggatgtatgaaagtttataaaatccccagtatagcgaccagcattc

SEQ ID NO: 30 - DNA, 84 bp, synthetic construct
  misc_feature 1..84: note = Primer
  source 1..84: mol_type = other DNA
atgagctcacaaccgtctttcgtcaccataaggggcaaggccatttctctagaaacacaaacgggacatggaggcccagaatac

SEQ ID NO: 31 - DNA, 113 bp, synthetic construct
  misc_feature 1..113: note = Primer
  source 1..113: mol_type = other DNA
agattcttgttatgtttttacatacgttgtttaataaaagtcgttatttatcagtggtgtggttgcttgtctggaattgggcttttccctgtgcagtatagcgaccagcattc

SEQ ID NO: 32 - DNA, 82 bp, synthetic construct
  misc_feature 1..82: note = Primer
  source 1..82: mol_type = other DNA
atgggtgaagattttatgcacccaccgtttcaaacgtacccttcaaagaacagcgaagggaagacatggaggcccagaatac

SEQ ID NO: 33 - DNA, 113 bp, synthetic construct
  misc_feature 1..113: note = Primer
  source 1..113: mol_type = other DNA
caaatttgtgcatatacttttcttgaccttattactcctcggcttgattatcattataaacactattccttctgttgcttggttaaaatgctacagtatagcgaccagcattc

SEQ ID NO: 34 - DNA, 92 bp, synthetic construct
  misc_feature 1..92: note = Primer
  source 1..92: mol_type = other DNA
atgacttcggctgtaccttatgatccatatgatgatctggataacaatccatttgctgagccccaggaggaagacatggaggcccagaatac

SEQ ID NO: 35 - DNA, 117 bp, synthetic construct
  misc_feature 1..117: note = Primer
  source 1..117: mol_type = other DNA
aaagatcaccttgttcaaaggtatgaattttctactttatatacgtattatcatgttcagaggatagatggattgactaagggtacagtacggcaaacagtatagcgaccagcattc

SEQ ID NO: 36 - DNA, 20 bp, synthetic construct
  misc_feature 1..20: note = Primer
  source 1..20: mol_type = other DNA
gacatggaggcccagaatac

SEQ ID NO: 37 - DNA, 20 bp, synthetic construct
  misc_feature 1..20: note = Primer
  source 1..20: mol_type = other DNA
cagtatagcgaccagcattc

SEQ ID NO: 38 - DNA, 117 bp, synthetic construct
  misc_feature 1..117: note = Primer
  source 1..117: mol_type = other DNA
ttctagaagatgagagaagagggaataatgagaaaggcgaaaaataaaggcacacaccatagcttcaaaatgtttctactccttttttactcttccagacatggaggcccagaatac

SEQ ID NO: 39 - DNA, 87 bp, synthetic construct
  misc_feature 1..87: note = Primer
  source 1..87: mol_type = other DNA
aagttaagtgcgcagaaagtaatatcatgcgtcaatcgtatgtgaatgctggtcgctatactggcacacaccatagcttcaaaatgt

SEQ ID NO: 40 - DNA, 24 bp, synthetic construct
  misc_feature 1..24: note = Primer
  source 1..24: mol_type = other DNA
gacaaacttggaatgtaaggcttc

SEQ ID NO: 41 - DNA, 118 bp, synthetic construct
  misc_feature 1..118: note = Primer
  source 1..118: mol_type = other DNA
attttttgttagacatataattttatatcattattcttattattcttataggaagtaccttcaaagaatggggtcttatcttgttttgcaagtaccacgacatggaggcccagaatac

SEQ ID NO: 42 - DNA, 88 bp, synthetic construct
  misc_feature 1..88: note = Primer
  source 1..88: mol_type = other DNA
aagttaagtgcgcagaaagtaatatcatgcgtcaatcgtatgtgaatgctggtcgctatactgggaagtaccttcaaagaatggggtc

SEQ ID NO: 43 - DNA, 25 bp, synthetic construct
  misc_feature 1..25: note = Primer
  source 1..25: mol_type = other DNA
tatctccaatgggttgctattcatc

SEQ ID NO: 44 - DNA, 118 bp, synthetic construct
  misc_feature 1..118: note = Primer
  source 1..118: mol_type = other DNA
gcattttgttgtgctgttacaaccacaacaaaacgaaaaacccgtatggatccaactggcaccgctggcttgaacaacaataccagccttccaacttcgacatggaggcccagaatac

SEQ ID NO: 45 - DNA, 84 bp, synthetic construct
  misc_feature 1..84: note = Primer
  source 1..84: mol_type = other DNA
aagttaagtgcgcagaaagtaatatcatgcgtcaatcgtatgtgaatgctggtcgctatactgtccaactggcaccgctggctt

SEQ ID NO: 46 - DNA, 117 bp, synthetic construct
  misc_feature 1..117: note = Primer
  source 1..117: mol_type = other DNA
acagcctcttgttgggcgaaaacagaggaggcgagcagcagggaggaccatgacaggacggcaccagcagaaaacttcattttgaatatgtattacttggttatggttatatatgac

SEQ ID NO: 47 - DNA, 118 bp, synthetic construct
  misc_feature 1..118: note = Primer
  source 1..118: mol_type = other DNA
gttcaatagtgtggttggtaaccaaattttctaggcgttgttgaaaataatcattagtgcccaccgtttgagcgtggtgtgacaccacgcccaagatagacatggaggcccagaatac

SEQ ID NO: 48 - DNA, 95 bp, synthetic construct
  misc_feature 1..95: note = Primer
  source 1..95: mol_type = other DNA
cccagatgcgaagttaagtgcgcagaaagtaatatcatgcgtcaatcgtatgtgaatgctggtcgctatactgtcattagtgcccaccgtttgag

SEQ ID NO: 49 - DNA, 99 bp, synthetic construct
  misc_feature 1..99: note = Primer
  source 1..99: mol_type = other DNA
attacgaaatggcctgtatgggtagattcttgttatgtttttacatacgttgtttaataaaagtcgttattcaattacaattcatcgtgaatggcatct

SEQ ID NO: 50 - DNA, 21 bp, synthetic construct
  misc_feature 1..21: note = Primer
  source 1..21: mol_type = other DNA
cccagatgcgaagttaagtgc

SEQ ID NO: 51 - DNA, 25 bp, synthetic construct
  misc_feature 1..25: note = Primer
  source 1..25: mol_type = other DNA
attacgaaatggcctgtatgggtag

SEQ ID NO: 52 - DNA, 118 bp, synthetic construct
  misc_feature 1..118: note = Primer
  source 1..118: mol_type = other DNA
tactcttgttcaatcagttagttatctttgttcaatagtgtggttggtaagcacacaccatagcttcaaaatgtttctactccttttttactcttccagacatggaggcccagaatac

SEQ ID NO: 53 - DNA, 97 bp, synthetic construct
  misc_feature 1..97: note = Primer
  source 1..97: mol_type = other DNA
cccagatgcgaagttaagtgcgcagaaagtaatatcatgcgtcaatcgtatgtgaatgctggtcgctatactggcacacaccatagcttcaaaatgt

SEQ ID NO: 54 - DNA, 100 bp, synthetic construct
  misc_feature 1..100: note = Primer
  source 1..100: mol_type = other DNA
attacgaaatggcctgtatgggtagattcttgttatgtttttacatacgttgtttaataaaagtcgttattcattacaattcatcgtgaatggcatcttc

SEQ ID NO: 55 - DNA, 20 bp, synthetic construct
  misc_feature 1..20: note = Primer
  source 1..20: mol_type = other DNA
ttaccacggtgctccagttg

SEQ ID NO: 56 - DNA, 20 bp, synthetic construct
  misc_feature 1..20: note = Primer
  source 1..20: mol_type = other DNA
aaccaagtcagcagcagaag

SEQ ID NO: 57 - DNA, 20 bp, synthetic construct
  misc_feature 1..20: note = Primer
  source 1..20: mol_type = other DNA
tgcggcacagaagagtaacc

SEQ ID NO: 58 - DNA, 20 bp, synthetic construct
  misc_feature 1..20: note = Primer
  source 1..20: mol_type = other DNA
ggcgataaacgataggcaac

SEQ ID NO: 59 - DNA, 22 bp, synthetic construct
  misc_feature 1..22: note = Primer
  source 1..22: mol_type = other DNA
tccgctaagaacaactaagtga

SEQ ID NO: 60 - DNA, 22 bp, synthetic construct
  misc_feature 1..22: note = Primer
  source 1..22: mol_type = other DNA
cactggctgtaaacggacctat

SEQ ID NO: 61 - DNA, 21 bp, synthetic construct
  misc_feature 1..21: note = Primer
  source 1..21: mol_type = other DNA
tgcaaacccagtgtaagacgc

SEQ ID NO: 62 - DNA, 22 bp, synthetic construct
  misc_feature 1..22: note = Primer
  source 1..22: mol_type = other DNA
atatggttcgagaacaggcatc

SEQ ID NO: 63 - DNA, 22 bp, synthetic construct
  misc_feature 1..22: note = Primer
  source 1..22: mol_type = other DNA
agcacgacatagaagtgaaacc

SEQ ID NO: 64 - DNA, 22 bp, synthetic construct
  misc_feature 1..22: note = Primer
  source 1..22: mol_type = other DNA
cgcaagggcaaacaggatagac

SEQ ID NO: 65 - DNA, 21 bp, synthetic construct
  misc_feature 1..21: note = Primer
  source 1..21: mol_type = other DNA
cgattgagtcgaacaccctga

SEQ ID NO: 66 - DNA, 19 bp, synthetic construct
  misc_feature 1..19: note = Primer
  source 1..19: mol_type = other DNA
cttgggtgcgtaggtctgg

SEQ ID NO: 67 - DNA, 22 bp, synthetic construct
  misc_feature 1..22: note = Primer
  source 1..22: mol_type = other DNA
gtcttgtaaccaatggcgaaac

SEQ ID NO: 68 - DNA, 20 bp, synthetic construct
  misc_feature 1..20: note = Primer
  source 1..20: mol_type = other DNA
gccaccacgattgacgaaca

SEQ ID NO: 69 - DNA, 22 bp, synthetic construct
  misc_feature 1..22: note = Primer
  source 1..22: mol_type = other DNA
ttaacagcgacttgcccacagg

SEQ ID NO: 70 - DNA, 21 bp, synthetic construct
  misc_feature 1..21: note = Primer
  source 1..21: mol_type = other DNA
agctagtctgtgacctgtacg

SEQ ID NO: 71 - DNA, 26 bp, synthetic construct
  misc_feature 1..26: note = Primer
  source 1..26: mol_type = other DNA
tgcacgtgataatatgttaccctgtc

SEQ ID NO: 72 - DNA, 26 bp, synthetic construct
  misc_feature 1..26: note = Primer
  source 1..26: mol_type = other DNA
ggaggaggatgagataagtagtttcc

SEQ ID NO: 73 - DNA, 22 bp, synthetic construct
  misc_feature 1..22: note = Primer
  source 1..22: mol_type = other DNA
aaaactctggcggctaaactgg

SEQ ID NO: 74 - DNA, 25 bp, synthetic construct
  misc_feature 1..25: note = Primer
  source 1..25: mol_type = other DNA
catcaatactggcgataagcgggac

SEQ ID NO: 75 - DNA, 27 bp, synthetic construct
  misc_feature 1..27: note = Primer
  source 1..27: mol_type = other DNA
tccttggactctttattcgacttcatc

SEQ ID NO: 76 - DNA, 22 bp, synthetic construct
  misc_feature 1..22: note = Primer
  source 1..22: mol_type = other DNA
cgcattataagtggtgtgccga

SEQ ID NO: 77 - DNA, 22 bp, synthetic construct
  misc_feature 1..22: note = Primer
  source 1..22: mol_type = other DNA
atgctgtgcttgggtgttttga

(19) United States
(12) Patent Application Publication
(10) Pub. No.: US 2025/0092436 A1
(43) Pub. Date: Mar. 20, 2025

(54) FUNGAL CELL WITH IMPROVED PROTEIN PRODUCTION CAPACITY

(71) Applicant: Melt&Marble AB, Göteborg (SE)

(72) Inventors: Mingtao Huang, Göteborg (SE); Anastasia Krivoruchko, Göteborg (SE); Florian David, Göteborg (SE); Jens Nielsen, Göteborg (SE)

(21) Appl. No.: 18/467,788

(22) Filed: Sep. 15, 2023

Related U.S. Application Data
(62) Division of application No. 16/634,267, filed on Jan. 27, 2020, now Pat. No. 11,795,487.

(30) Foreign Application Priority Data
Jul. 31, 2017 (SE) 1750968-8

(51) Int. Cl. C12P 21/02; C12N 1/18

(52) U.S. Cl. CPC C12P 21/02; C12N 1/185

(57) ABSTRACT

The present invention related to the provision of genetically modified fungal cells, such as yeast cells with an improved ability for producing and secreting different recombinant proteins. The improved ability is obtained by disruption in intracellular transport between the Golgi and the endosome. In particular embodiments, the disruption is achieved by downregulation or deletion of the gene encoding a Tda3p homolog. The fungal cell and method of the invention would allow for large-scale production of recombinant proteins in fungal cells.

### STATEMENT OF PRIORITY

This application is a divisional of and claims priority to U.S. patent application Ser. No. 16/634,267, filed on Jan. 27, 2020, which is a 35 U.S.C. § 371 national phase application of International Application Serial No. PCT/SE2018/050779, filed Jul. 30, 2018, which claims the benefit, under 35 U.S.C. § 119(a), of Swedish Patent Application No. 1750968-8; filed Jul. 31, 2017, the entire contents of which are incorporated by reference herein.

### STATEMENT REGARDING ELECTRONIC FILING OF A SEQUENCE LISTING

A Sequence Listing in XML text format, submitted under 37 C.F.R. § 1.821-1.834, entitled 9737-77DV_ST26.xml, 104,873 bytes in size, generated on Sep. 13, 2023 and filed electronically, is provided in lieu of a paper copy. This Sequence Listing is hereby incorporated herein by reference into the specification for its disclosures.

### TECHNICAL FIELD

The present invention relates generally to the development of genetically engineered microorganisms. More specifically the invention relates to fungal cells containing modifications allowing production and secretion of high levels of recombinant proteins.

### BACKGROUND

Production of recombinant proteins by fungal cells plays an important role in various industries. For example, various industrial enzymes and biopharmaceuticals are produced by fungal systems. It is therefore desirable to develop fungal platform strains that are able to produce high levels of different recombinant proteins.

Various research efforts have been aimed at creating platform strains for protein production via genetic engineering of the host cell. For example, several studies have aimed to increase recombinant protein production by increasing the protein folding capacity of the cell. Tang et al (Biotechnol Bioeng. 2015 September; 112(9):1872-82. doi:10.1002/bit.25596) over-expressed the endoplasmic reticulum (ER) chaperone protein BiP and the disulfide isomerase Pdi1p in yeast, and thereby managed to increase the secretion of three heterologous proteins, β-glucosidase, endoglucanase, and a-amylase. In addition Hou et al (Appl Microbiol Biotechnol. 2013 April; 97(8):3559-68. doi:10.1007/s00253-012-4596-9.) showed that overexpression of HSF1, a transcription factor that controls the expression of multiple protein chaperones, led to increased production of heterologous α-amylase, endogenous invertase and human insulin precursor. Another transcription factor, HAC1, is involved in the general unfolded protein response. It facilitates the expression of PDI or BiP in a cell, and has been successfully employed to obtain improved levels of production of antibodies by Gasser et al (Biotechnol Bioeng. 2006 Jun. 5; 94(2):353-61.). Koskela et al (Biotechnol J. 2017 Apr. 21. doi:10.1002/biot.201600631.) showed that expression of mammalian BiP, the co-chaperone GRP170, or the peptidyl-prolyl isomerase FKBP2 increased antibody production in yeast. De Ruijter et al (Microb Cell Fact. 2016 May 23; 15:87. doi:10.1186/s12934-016-0488-5.) have also shown that overexpression of the folding factor Cpr5p could lead to increased antibody production. Other studies have focused on proteases and showed that deletion of proteases could lead to increased protein production. Tomimoto et al (Biosci Biotechnol Biochem. 2013; 77:2461-6) were able to obtain higher production of human interferon-β in yeast by disruption of the proteases encoded by PEP4 and PRB1. Furthermore, Choo et al (J Biotechnol. 2010 Aug. 20; 149:1-7. doi:10.1016/j.jbiotec.2010.06.014.) showed that disruption of various yapsin proteases reduced proteolytic degradation of human parathyroid hormone protein during fermentation.

Other studies have involved engineering of intracellular trafficking. Hou et al (Metab Eng. 2012 March; 14(2):120-7. doi:10.1016/j.ymben.2012.01.002) have shown that overexpression of Seclp, a protein that is involved in exocytosis in S. cerevisiae, led to increased secretion of heterologous proteins human insulin precursor and α-amylase, and also the secretion of an endogenous protein invertase around 1.5×.

Other studies have also attempted to engineer the components involved in vesicle trafficking from the endoplasmic reticulum (ER) to the Golgi, and from the Golgi to the plasma membrane (PM). Bao et al (Appl Environ Microbiol. 2017. 5. pii: AEM.03400-16. doi:10.1128/AEM.03400-16.) have shown that overexpression of Sec16p, a protein involved in transport between the ER and the Golgi, led to increased secretion of heterologous α-amylase. Tang et al (Biotechnol Biofuels. 2017 Feb. 27; 10:53. doi:10.1186/s13068-017-738-8.) showed that engineering the targeted components in the ER to Golgi vesicle trafficking, including Sec12p, Sec13p, Erv25p and Bos1p, enhanced the extracellular activity of heterologous endoglucanase. In addition, over-expression of the components in the Golgi to plasma membrane vesicle trafficking, including Sso1p, Snc2p, Sec1p, Exo70p, Ypt32p and Sec4p, led to increased secretion of β-glucosidase. Van Zyl et al (Appl Microbiol Biotechnol. 2016 January; 100:505-18. doi:10.1007/s00253-015-7022-2.) have also demonstrated that production of heterologous cellobiohydrolase and β-glucosidase could be increased by single and co-overexpression of some of the endoplasmic reticulum (ER)-to-Golgi SNAREs (BOS1, BET1, SEC22 and SED5). Furthermore, the patent application US 2013/0011875 A1, discloses a Pichia pastoris cell with disrupted vacuolar sorting activity, wherein the disruption occurs through deletion of vacuolar protein sorting receptor 10 (Vps10), as well as disruption of one or more genes that encode a protein associated with recycling of Vps10 to the late Golgi.

A study by Huang et al (Proc Natl Acad Sci U S A. 2015 Aug. 25; 112(34):E4689-96. doi:10.1073/pnas.1506460112.) reported combination of UV mutagenesis and microfluidic sorting to uncover potential targets and reported that deletion of HDA2, HDA3 and SNC2 in yeast results in increased protein production.

TDA3 (also known as BTN3) is a putative oxidoreductase and was shown to interact with both epsins Ent3 and Ent5. TDA3 is a negative regulator of the Batten-disease-linked protein Btn2 involved in the retrieval of specific SNAREs (Vti1, Snc1, Tlg1 and Tlg2) from the late endosome to the Golgi. It was suggested that TDA3 sequesters Btn2 away from its substrates, thus down-regulating protein trafficking and aggregation. It was shown that in btn34 mutant cells, endosomal sorting of ubiquitylated cargos and endosomal recycling of the Snc1 SNARE are delayed.

COG5 is a component of the conserved oligomeric Golgi complex that functions in protein trafficking to mediate fusion of transport vesicles to Golgi compartments

### SUMMARY

It is a general objective to provide an improved fungal cell.

It is a particular objective to provide a fungal cell that can be used for fermentation-based production of recombinant proteins.

These and other objectives are met by embodiments as disclosed herein.

An aspect of the embodiments relates to a fungal cell. According to the embodiments, the fungal cell lacks a gene encoding Tda3p or comprises a disrupted endogenous gene encoding Tda3p. The fungal cell also comprises a gene encoding a recombinant protein.

Another aspect of the embodiments relates to a method for producing a recombinant protein. The method comprises culturing a fungal cell according to any of the embodiments in a culture medium and in culture conditions suitable for production of the recombinant protein by the fungal cell. The method also comprises collecting the recombinant protein from the culture medium and/or from the fungal cell.

The fungal cell of the embodiments comprises modifications to intracellular transport between the Golgi and the endosome, combined with expression of a recombinant protein.

### BRIEF DESCRIPTION OF THE DRAWINGS

The embodiments, together with further objects and advantages thereof, may best be understood by making reference to the following description taken together with the accompanying drawings, in which:

FIG. 1: Overview of the intracellular trafficking in a fungal cell. Proteins are transported from the endoplasmic reticulum (ER) to the Golgi, where they are sorted into anterograde transport vesicles for ER resident proteins, into secretory vesicles for plasma membrane (PM) and secretion, and into vacuolar protein sorting vesicles for vacuolar proteins passing through the endosomes. The present invention involves disruption of transport between the Golgi and the endosome.

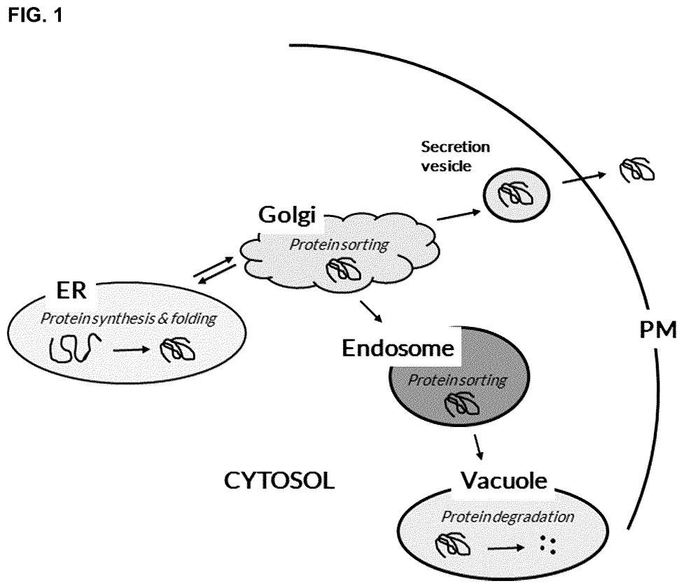

FIG. 2: Disruption of selected genes, especially HDA2, HDA3, PGM2, PXA1, EMC1, GOS1, VPS5, TDA3 or SNC2 in the yeast Saccharomyces cerevisiae leads to increase in recombinant protein production. a) Recombinant protein yield. b) Intracellular percentage-the fraction of the protein that is retained in the cell c) Dry cell weight. α-amylase was used as a model protein in this study.

FIG. 3: Combinatorial effect of gene deletions on protein production in yeast. a) Recombinant protein yield. b) Intracellular percentage—the fraction of the protein that is retained in the cell. c) Dry cell weight. α-amylase was used as a model protein in this study.

FIG. 4: Disruption of VPS17 increases recombinant protein production a) and decreases intracellular recombinant protein percentage b), suggesting increased secretion. α-amylase was used as a model protein in this study.

FIG. 5: Effect of ERV29 and/or COG5 overexpression on total protein yield a). intracellular protein percentage b) and dry cell weight c). α-amylase was used as a model protein in this study.

FIG. 6: Combination of selected gene disruption and overexpression increases protein production. a) Total protein yield. b) Intracellular protein percentage. c) Dry cell weight. α-amylase was used as a model protein in this study. The figure shows that combination of deletions of HDA2, VPS5, GOS1 and TDA3 with overexpression of COG5 and PDI1 results in increased α-amylase protein production and decreased intracellular α-amylase percentage, suggesting increased secretion.

FIG. 7: Combination of selected modifications with additional recombinant proteins. This example shows that the best-producing strain (containing deletions in HDA2, VPS5, GOS1 and TDA3 and overexpression of COG5 and PDI1) can also be used for increased production of glucan 1,4-α-glucosidase.

FIG. 8: Fed-batch fermentation of the best-producing strain containing deletions in HDA2, VPS5, GOS1 and TDA3 and overexpression of COG5 and PDI1. α-amylase was used as a model protein in this study.

### DETAILED DESCRIPTION OF THE INVENTION AND PREFERRED EMBODIMENTS THEREOF

The present invention now will be described hereinafter with reference to the accompanying drawings and examples, in which embodiments of the invention are shown. This description is not intended to be a detailed catalogue of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined herein, scientific and technical terms used herein will have the meanings that are commonly understood by those of ordinary skill in the art.

Generally, nomenclatures used in connection with techniques of biochemistry, enzymology, molecular and cellular biology, microbiology, genetics and protein and nucleic acid chemistry and hybridization, described herein, are those well-known and commonly used in the art.

Conventional methods and techniques mentioned herein are explained in more detail, for example, in Molecular Cloning, a laboratory manual [second edition] Sambrook et al. Cold Spring Harbor Laboratory, 1989, for example in Sections 1.21 “Extraction And Purification Of Plasmid DNA”, 1.53 “Strategies For Cloning In Plasmid Vectors”, 1.85 “Identification Of Bacterial Colonies That Contain Recombinant Plasmids”, 6 “Gel Electrophoresis Of DNA”, 14 “In vitro Amplification Of DNA By The Polymerase Chain Reaction”, and 17 “Expression Of Cloned Genes In Escherichia coli” thereof.

Enzyme Commission (EC) numbers (also called “classes” herein), referred to throughout this specification, are according to the Nomenclature Committee of the International Union of Biochemistry and Molecular Biology (NC-IUBMB) in its resource “Enzyme Nomenclature” (1992, including Supplements 6-17) available, for example, as “Enzyme nomenclature 1992: recommendations of the Nomenclature Committee of the International Union of Biochemistry and Molecular Biology on the nomenclature and classification of enzymes”, Webb, E. C. (1992), San Diego: Published for the International Union of Biochemistry and Molecular Biology by Academic Press (ISBN 0-12-227164-5). This is a numerical classification scheme based on the chemical reactions catalyzed by each enzyme class.

The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

As used herein, the transitional phrase “consisting” essentially of means that the scope of a claim is to be interpreted to encompass the specified materials or steps recited in the claim and those that do not materially affect the basic and novel characteristic(s) of the claimed invention. Thus, the term “consisting essentially of when used in a claim of this invention is not intended to be interpreted to be equivalent to “comprising.”

To facilitate understanding of the invention, a number of terms are defined below.

Also as used herein, the terms “nucleotide sequence” “nucleic acid,” “nucleic acid molecule,” “oligonucleotide” and “polynucleotide” refer to RNA or DNA, including cDNA, a DNA fragment or portion, genomic DNA, synthetic DNA, plasmid DNA, mRNA, and anti-sense RNA, any of which can be single stranded or double stranded, linear or branched, or a hybrid thereof. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein the term “recombinant” when applied to nucleic acid means that a particular nucleic acid (DNA or RNA) is the product of various combinations of fusion, cloning, restriction, genetic recombination and/or ligation steps resulting in a construct having a structural coding or non-coding sequence distinguishable from endogenous nucleic acids found in natural systems. The term “recombinant protein” refers to protein that can result from the expression of recombinant DNA within a cell.

As used herein, the term “gene” refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions, e.g. introns, regulatory elements, promoters, enhancers, termination sequences and/or 5′ and 3′ untranslated regions. A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

A “disrupted gene” as defined herein involves any mutation or modification to a gene resulting in a partial or fully non-functional gene and gene product. Such a mutation or modification includes, but is not limited to, a missense mutation, a nonsense mutation, a deletion, a substitution, an insertion, addition of a targeting sequence and the like. Furthermore, a disruption of a gene can be achieved also, or alternatively, by mutation or modification of control elements controlling the transcription of the gene, such as mutation or modification in a promoter, terminator and/or enhancement elements. In such a case, such a mutation or modification results in partially or fully loss of transcription of the gene, i.e. a lower or reduced transcription as compared to native and non-modified control elements. As a result a reduced, if any, amount of the gene product will be available following transcription and translation. Furthermore, disruption of a gene could also entail adding or removing a localization signal from the gene, resulting in decreased presence of the gene product in its native subcellular compartment.

The objective of gene disruption is to reduce the available amount of the gene product, including fully preventing any production of the gene product, or to express a gene product that lacks or having lower enzymatic activity as compared to the native or wild type gene product.

A “codon optimized” version of a gene refers to an exogenous gene introduced into a cell and where the codons of the gene have been optimized with regard to the particular cell. Generally, not all tRNAs are expressed equally or at the same level across species. Codon optimization of a gene sequence thereby involves changing codons to match the most prevalent tRNAs, i.e. to change a codon recognized by a low prevalent tRNA with a synonymous codon recognized by a tRNA that is comparatively more prevalent in the given cell. This way the mRNA from the codon optimized gene will be more efficiently translated. The codon and the synonymous codon preferably encode the same amino acid.

As used herein, the term “allele” refers to a variant form of a given gene. This can include a mutated form of a gene where one or more of the amino acids encoded by the gene have been removed or substituted by a different amino acid.

As used herein, the terms “peptide”, “polypeptide”, and “protein” are used interchangeably to indicate to a polymer of amino acid residues. The terms “peptide”, “polypeptide” and “protein” also includes modifications including, but not limited to, lipid attachment, glycosylation, glycosylation, sulfation, hydroxylation, γ-carboxylation of L-glutamic acid residues and ADP-ribosylation.

As used herein, the term “enzyme” is defined as a protein which catalyzes a chemical or a biochemical reaction in a cell. Usually, according to the present invention, the nucleotide sequence encoding an enzyme is operably linked to a nucleotide sequence (promoter) that causes sufficient expression of the corresponding gene in the cell to confer to the cell the ability to produce desired metabolites.

As used herein, the term “open reading frame (ORF)” refers to a region of RNA or DNA encoding polypeptide, a peptide, or protein.

As used herein, the term “genome” encompasses both the plasmids and chromosomes in a host cell. For instance, encoding nucleic acids of the present disclosure which are introduced into host cells can be portion of the genome whether they are chromosomally integrated or plasmids-localized.

As used herein, the term “promoter” refers to a nucleic acid sequence which has functions to control the transcription of one or more genes, which is located upstream with respect to the direction of transcription of the transcription initiation site of the gene. Suitable promoters in this context include both constitutive and inducible natural promoters as well as engineered promoters, which are well known to the person skilled in the art.

Suitable promoters for use in fungal cells may be the promoters of PDC, GPD1, TEF1, PGK1 and TDH. Other suitable promoters include the promoters of GAL1, GAL2, GAL10, GAL7, CUP1, HIS3, CYC1, ADH1, PGL, GAPDH, ADC1, URA3, TRP1, LEU2, TP1, AOX1 and ENO1.

As used herein, the term “terminator” refers to a “transcription termination signal” if not otherwise noted. Terminators are sequences that hinder or stop transcription of a polymerase.

As used herein, “recombinant fungal cells” according to the present disclose is defined as cells which contain additional copies or copy of an endogenous nucleic acid sequence or are transformed or genetically modified with polypeptide or a nucleotide sequence that does not naturally occur in the fungal cells. The wildtype fungal cells are defined as the parental cells of the recombinant fungal cells, as used herein.

As used herein, the terms “increase,” “increases,” “increased,” “increasing,” “enhance,” “enhanced,” “enhancing,” and “enhancement” (and grammatical variations thereof) indicate an elevation of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more, or any range therein, as compared to a control.

As used herein, the terms “reduce,” “reduces,” “reduced,” “reduction,” “diminish,” “suppress,” and “decrease” and similar terms mean a decrease of at least about, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more, or any range therein, as compared to a control.

A “reduced expression” of a gene as used herein involves a genetic modification that reduces the transcription of the gene, reduces the translation of the mRNA transcribed from the gene and/or reduces post-translational processing of the protein translated from the mRNA. Such genetic modification includes insertion(s), deletion(s), replacement s) or mutation(s) applied to the control sequence, such as a promoter and enhancer, of the gene. For instance, the promoter of the gene could be replaced by a less active or inducible promoter to thereby result in a reduced transcription of the gene. Also a knock-out of the promoter would result in reduced, typically zero, expression of the gene.

As used herein the terms “knock-out” or “deletion” or “disruption” refers to a gene that is inoperative or knocked out and/or a nonfunctional gene product, e.g. a polypeptide having essentially no activity, e.g. less than about 10% or even 5% as compared to the activity of the wild type polypeptide.

As used herein, the term “portion” or “fragment” of a nucleotide sequence of the invention will be understood to mean a nucleotide sequence of reduced length relative to a reference nucleic acid or nucleotide sequence and comprising, consisting essentially of and/or consisting of a nucleotide sequence of contiguous nucleotides identical or almost identical, e.g. 80%, 81%, 82%, 83%, 84%, 85%, 86%, 87%, 88%, 89%, 90%, 91%, 92%, 93%, 94%, 95%, 96%, 98%, 99% identical, to the reference nucleic acid or nucleotide sequence. Such a nucleic acid fragment or portion according to the invention may be, where appropriate, included in a larger polynucleotide of which it is a constituent.

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity, i.e. sequence similarity or identity. Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity, e.g. at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, and/or 100%, to said nucleotide sequence.

The term “overexpress,” “overexpresses” or “overexpression” as used herein refers to higher levels of activity of a gene, e.g. transcription of the gene; higher levels of translation of mRNA into protein; and/or higher levels of production of a gene product, e.g. polypeptide, than would be in the cell in its native or control, e.g. not transformed with the particular heterologous or recombinant polypeptides being overexpressed, state. A typical example of an overexpressed gene is a gene under transcription control of another promoter as compared to the native promoter of the gene. Also, or alternatively, other changes in the control elements of a gene, such as enhancers, could be used to overexpress the particular gene. Furthermore, modifications that affect, i.c. increase, the translation of the mRNA transcribed from the gene could, alternatively or in addition, be used to achieve an overexpressed gene as used herein. These terms can also refer to an increase in the number of copies of a gene and/or an increase in the amount of mRNA and/or gene product in the cell. Overexpression can result in levels that are 25%, 50%, 100%, 200%, 500%, 1000%, 2000% or higher in the cell, or any range therein, as compared to control levels.

As used herein, the terms “exogenous” or “heterologous” when used with respect to a nucleic acid (RNA or DNA), protein or gene refer to a nucleic acid, protein or gene which occurs non-naturally as part of the cell, organism, genome, RNA or DNA sequence into which it is introduced, including non-naturally occurring multiple copies of a naturally occurring nucleotide sequence. Such an exogenous gene could be a gene from another species or strain, a modified, mutated or evolved version of a gene naturally occurring in the host cell or a chimeric version of a gene naturally occurring in the host cell or fusion genes. In these former cases, the modification, mutation or evolution causes a change in the nucleotide sequence of the gene to thereby obtain a modified, mutated or evolved gene with another nucleotide sequence as compared to the gene naturally occurring in the host cell. Evolved gene refers to genes encoding evolved genes and obtained by genetic modification, such as mutation or exposure to an evolutionary pressure, to derive a new gene with a different nucleotide sequence as compared to the wild type or native gene. A chimeric gene is formed through the combination of portions of one or more coding sequences to produce a new gene. These modifications are distinct from a fusion gene, which merges whole gene sequences into a single reading frame and often retain their original functions.

An “endogenous”, “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in or endogenous to the organism. A “homologous” nucleic acid sequence is a nucleotide sequence naturally associated with a host cell into which it is introduced.

As used herein, the term “modified”, when it is used with respect to an organism, refers to a host organism that has been modified to increase production of proteins, as compared with an otherwise identical host organism that has not been so modified. In principle, such “modification” in accordance with the present disclosure may comprise any physiological, genetic, chemical, or other modification that appropriately alters production of proteins in a host organism as compared with such production in an otherwise identical organism which is not subject to the said modification. In most of the embodiments, however, the modification will comprise a genetic modification. In certain embodiments, as described herein, the modification comprises introducing genes into a host cell, and particularly into a host cell which is disrupted in the Golgi-endosome trafficking. In some embodiments, a modification comprises at least one physiological, chemical, genetic, or other modification; in other embodiments, a modification comprises more than one chemical, genetic, physiological, or other modification. In certain aspects where more than one modification is made use of, such modifications can include any combinations of physiological, genetic, chemical, or other modification (e.g., one or more genetic, chemical and/or physiological modification(s)). Genetic modifications which boost the activity of a polypeptide include, but are not limited to: introducing one or more copies of a gene encoding the polypeptide (which may distinguish from any gene already present in the host cell encoding a polypeptide having the same activity); altering a gene present in the cell to increase transcription or translation of the gene (e.g., altering, adding additional sequence to, replacement of one or more nucleotides, deleting sequence from, or swapping for example, regulatory, a promoter or other sequence); and altering the sequence (e.g. non-coding or coding) of a gene encoding the polypeptide to boost activity (e.g., by increasing enzyme activity, decrease feedback inhibition, targeting a specific subcellular location, boost mRNA stability, boost protein stability). Genetic modifications that reduce activity of a polypeptide include, but are not limited to: deleting a portion or all of a gene encoding the polypeptide; inserting a nucleic acid sequence which disrupts a gene encoding the polypeptide; changing a gene present in the cell to reduce transcription or translation of the gene or stability of the mRNA or polypeptide encoded by the gene (for example, by adding additional sequence to, altering, deleting sequence from, replacement of one or more nucleotides, or swapping for example, replacement of one or more nucleotides, a promoter, regulatory or other sequence).

The term “overproducing” is used herein in reference to the production of proteins in a host cell and indicates that the host cell is producing more of protein by virtue of the introduction of nucleic acid sequences which encode different polypeptides involved in the host cell's metabolic pathways or as a result of other modifications as compared with the unmodified host cell or wild-type cell.

As used herein, the term “secretion” or “secreting” refers to the excretion of material, such as proteins from the cell.

As used herein, the term “flux”, “metabolic flux” or “carbon flux” refers to the rate of turnover of molecules through a given reaction or a set of reactions. Flux in a metabolic pathway is regulated by the enzymes involved in the pathway. Pathways or reactions characterized by a state of increased flux compared to a control have an increased rate of generation of products from given substrates. Pathways or reactions characterized by a state of decreased flux compared to a control have a decreased rate of generation of products from given substrates. Flux towards products of interest can be increased by removing or decreasing competitive reactions or by increasing the activities of enzymes involved in generation of said products.

As used herein the term “vector” is defined as a linear or circular DNA molecule comprising a polynucleotide encoding a polypeptide of the invention, and which is operably linked to additional nucleotides that ensure its expression.

“Introducing” in the context of a yeast cell means contacting a nucleic acid molecule with the cell in such a manner that the nucleic acid molecule gains access to the interior of the cell. Accordingly, polynucleotides and/or nucleic acid molecules can be introduced yeast cells in a single transformation event, in separate transformation events. Thus, the term “transformation” as used herein refers to the introduction of a heterologous nucleic acid into a cell. Transformation of a yeast cell can be stable or transient.

“Transient transformation” in the context of a polynucleotide means that a polynucleotide is introduced into the cell and does not integrate into the genome of the cell.

By “stably introducing” or “stably introduced” in the context of a polynucleotide introduced into a cell, it is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear genome. Stable transformation as used herein can also refer to a nucleic acid molecule that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more nucleic acid molecules introduced into an organism. Stable transformation of a cell can be detected by, for example, a Southern blot hybridization assay of genomic DNA of the cell with nucleic acid sequences which specifically hybridize with a nucleotide sequence of a nucleic acid molecule introduced into an organism (e.g., a yeast). Stable transformation of a cell can be detected by, for example, a Northern blot hybridization assay of RNA of the cell with nucleic acid sequences which specifically hybridize with a nucleotide sequence of a nucleic acid molecule introduced into a yeast or other organism. Stable transformation of a cell can also be detected by, e.g., a polymerase chain reaction (PCR) or other amplification reaction as are well known in the art, employing specific primer sequences that hybridize with target sequence(s) of a nucleic acid molecule, resulting in amplification of the target sequence(s), which can be detected according to standard methods Transformation can also be detected by direct sequencing and/or hybridization protocols well known in the art.

Embodiments of the present invention also encompass variants of the polypeptides as defined herein. As used herein, a “variant” means a polypeptide in which the amino acid sequence differs from the base sequence from which it is derived in that one or more amino acids within the sequence are substituted for other amino acids. For example, a variant of SEQ ID NO:1 may have an amino acid sequence at least about 50% identical to SEQ ID NO:1, for example, at least about 55%, 60%, 65%, 70%, 75%, 80%, 85%, 90%, 91%, 92%, 93%, 94%, 95%, 96%, 97%, 98%, 99% or about 100% identical. The variants and/or fragments are functional variants/fragments in that the variant sequence has similar or identical functional enzyme activity characteristics to the enzyme having the non-variant amino acid sequence specified herein (and this is the meaning of the term “functional variant” as used throughout this specification).

A “functional variant” or “functional fragment” of any of the above amino acid sequences, therefore, is any amino acid sequence which remains within the same enzyme category (i.c., has the same EC number) as the non-variant sequences. Methods of determining whether an enzyme falls within a particular category are well known to the skilled person, who can determine the enzyme category without use of inventive skill. Suitable methods may, for example, be obtained from the International Union of Biochemistry and Molecular Biology.

Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows:

Class Amino Acid Examples
Nonpolar: A, V, L, I, P, M, F, W
Uncharged polar: G, S, T, C, Y, N, Q
Acidic: D, E
Basic: K, R, H.

As is well known to those skilled in the art, altering the primary structure of a polypeptide by a conservative substitution may not significantly alter the activity of that polypeptide because the side-chain of the amino acid which is inserted into the sequence may be able to form similar bonds and contacts as the side chain of the amino acid which has been substituted out. This is so even when the substitution is in a region which is critical in determining the polypeptide's conformation.

In embodiments of the present invention, non-conservative substitutions are possible provided that these do not interrupt the enzyme activities of the polypeptides, as defined elsewhere herein. The substituted versions of the enzymes must retain characteristics such that they remain in the same enzyme class as the non-substituted enzyme, as determined using the NC-IUBMB nomenclature discussed above.

Broadly speaking, fewer non-conservative substitutions than conservative substitutions will be possible without altering the biological activity of the polypeptides. Determination of the effect of any substitution (and, indeed, of any amino acid deletion or insertion) is wholly within the routine capabilities of the skilled person, who can readily determine whether a variant polypeptide retains the enzyme activity according to aspects of the invention. For example, when determining whether a variant of the polypeptide falls within the scope of the invention (i.e., is a “functional variant or fragment” as defined above), the skilled person will determine whether the variant or fragment retains the substrate converting enzyme activity as defined with reference to the NC-IUBMB nomenclature mentioned elsewhere herein. All such variants are within the scope of the invention.

Using the standard genetic code, further nucleic acid sequences encoding the polypeptides may readily be conceived and manufactured by the skilled person, in addition to those disclosed herein. The nucleic acid sequence may be DNA or RNA, and where it is a DNA molecule, it may for example comprise a cDNA or genomic DNA. The nucleic acid may be contained within an expression vector, as described elsewhere herein.

Embodiments of the invention, therefore, encompass variant nucleic acid sequences encoding the polypeptides contemplated by embodiments of the invention. The term “variant” in relation to a nucleic acid sequence means any substitution of, variation of, modification of, replacement of, deletion of, or addition of one or more nucleotide(s) from or to a polynucleotide sequence, providing the resultant polypeptide sequence encoded by the polynucleotide exhibits at least the same or similar enzymatic properties as the polypeptide encoded by the basic sequence. The term includes allelic variants and also includes a polynucleotide (a “probe sequence”) which substantially hybridizes to the polynucleotide sequence of embodiments of the present invention. Such hybridization may occur at or between low and high stringency conditions. In general terms, low stringency conditions can be defined as hybridization in which the washing step takes place in a 0.330-0.825 M NaCl buffer solution at a temperature of about 40-48° C. below the calculated or actual melting temperature (Tm) of the probe sequence (for example, about ambient laboratory temperature to about 55° C.), while high stringency conditions involve a wash in a 0.0165-0.0330 M NaCl buffer solution at a temperature of about 5-10° C. below the calculated or actual Tm of the probe sequence (for example, about 65° C.). The buffer solution may, for example, be SSC buffer (0.15M NaCl and 0.015M tri-sodium citrate), with the low stringency wash taking place in 3×SSC buffer and the high stringency wash taking place in 0.1×SSC buffer. Steps involved in hybridization of nucleic acid sequences have been described for example in Molecular Cloning, a laboratory manual [second edition] Sambrook et al. Cold Spring Harbor Laboratory, 1989, for example in Section 11 “Synthetic Oligonucleotide Probes” thereof (herein incorporated by reference)

Preferably, nucleic acid sequence variants have about 55% or more of the nucleotides in common with the nucleic acid sequence of embodiments of the present invention, more preferably at least 60%, 65%, 70%, 80%, 85%, or even 90%, 95%, 98% or 99% or greater sequence identity.

Variant nucleic acids of the invention may be codon-optimized for expression in a particular host cell.

As used herein, “sequence identity” refers to sequence similarity between two nucleotide sequences or two peptide or protein sequences. The similarity is determined by sequence alignment to determine the structural and/or functional relationships between the sequences.

Sequence identity between amino acid sequences can be determined by comparing an alignment of the sequences using the Needleman-Wunsch Global Sequence Alignment Tool available from the National Center for Biotechnology Information (NCBI), Bethesda, Md., USA, for example via http://blast.ncbi.nlm.nih.gov/Blast.cgi, using default parameter settings (for protein alignment, Gap costs Existence:11 Extension:1). Sequence comparisons and percentage identities mentioned in this specification have been determined using this software. When comparing the level of sequence identity to, for example, SEQ ID NO:1, this, preferably should be done relative to the whole length of SEQ ID NO:1 (i.c., a global alignment method is used), to avoid short regions of high identity overlap resulting in a high overall assessment of identity. For example, a short polypeptide fragment having, for example, five amino acids might have a 100% identical sequence to a five amino acid region within the whole of SEQ ID NO:1, but this does not provide a 100% amino acid identity unless the fragment forms part of a longer sequence which also has identical amino acids at other positions equivalent to positions in SEQ ID NO:1. When an equivalent position in the compared sequences is occupied by the same amino acid, then the molecules are identical at that position. Scoring an alignment as a percentage of identity is a function of the number of identical amino acids at positions shared by the compared sequences. When comparing sequences, optimal alignments may require gaps to be introduced into one or more of the sequences, to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties. As mentioned above, the percentage sequence identity may be determined using the Needleman-Wunsch Global Sequence Alignment tool, using default parameter settings. The Needleman-Wunsch algorithm was published in J. Mol. Biol. (1970) vol. 48:443-53.

An aspect of the embodiments relates to a fungal cell. According to the embodiments, the fungal cell lacks a gene encoding Tda3p or comprises a disrupted endogenous gene encoding Tda3p. The fungal cell also comprises a gene encoding a recombinant protein.

The present embodiments are based on engineering of intracellular trafficking as a means of increasing recombinant protein production in fungal cells. We surprisingly found that by disrupting the transport between the Golgi and the endosome, specifically by disrupting the protein Tda3p, optionally in combination with other targets, such as Goslp, it was possible to increase recombinant protein production and secretion several fold in the fungal cell.

Preferred features of each aspect of the invention may be as described in connection with any of the other aspects. Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

In the following, various embodiments of the present invention will be described in more detail.

Preferably, the fungal cell to be modified can be selected from any known genus and species of fungus. In one embodiment, the fungal cell is selected from a group consisting of Saccharomyces, Kluyveromyces, Zygosaccharomyces, Candida, Hansenula, Torulopsis, Kloeckera, Pichia, Schizosaccharomyces, Trigonopsis, Brettanomyces, Debaromyces, Nadsonia, Lipomyces, Cryptococcus, Aureobasidium, Trichosporon, Lipomyces, Rhodotorula, Yarrowia, Rhodosporidium, Phaffia, Schwanniomyces, Aspergillus, and Ashbya,. Saccharomyces cerevisiae is commonly used yeast in industrial processes, but the disclosure is not limited thereto. Other yeast species useful in the present disclosure include but are not limited to Pichia pastoris, Ashbya gossypii, Saccharomyces boulardii, Zygosaccharomyces bailii, Kluyveromyces lactis, Rhodosporidium toruloides and Yarrowia lipolytica.

In some embodiments, the transport between the Golgi and the endosome is disrupted in the fungal cell. The modification(s) allow(s) for increased production and/or secretion of recombinant proteins in the fungal cell. This can be achieved by downregulation of proteins involved in the transport between the Golgi and the endosome. For example, in S. cerevisiae, this includes the proteins Tda3p, Gos1p, Vps5p, Vps17p, Vps10p, Ccz1p, Hse1p, Pep8p, Vps29p, Vps35p, Snx41p, Btn2p, Dop1p, Trs130p, Trs120p, Snx4p, Fab1p, Ypt7p, Ent5p, Vps53p, Laa1p, Atg20p, Vps52p, Sft1p, Sft2p, Vps51p, Ent3p, Snx3p, Ypt31p, Vps54p, Ypt32p, Ykt6p Mvp1p, Tlg1p, Trs65p, Tca17p, Rcy1p, Ypt6p, Vti1p, Rgp1p, Ric1p, Got1p, Rhb1p, Gga1p, Gga2p, Mon2p, Vta1p and Vps45p. These proteins are encoded by the genes TDA3, GOS1, VPS5, VPS17, VPS10, CCZ1, HSE1, PEP8, VPS29, VPS35, SNX41, BTN2, DOP1, TRS130, TRS120, SNX4, FAB1, YPT7, ENT5, YKR078W, VPS53, LAA1, ATG20, VPS52, SFT1, SFT2, VPS51, ENT3, SNX3, YPT31, VPS54, YPT32, YKT6, MVP1, TLG1, TLG2, TRS65, TCA17, RCY1, YPT6, VTI1, RGP1, RIC1, GOT1, RHB, GGA1, GGA2, MON2, VTA1 and VPS45, respectively.

In a preferred embodiment, the modifications to the transport between the Golgi and the endosome involve disruption of Tda3p (SEQ ID NO: 1). This could be achieved by deleting the endogenous gene coding for this protein. For example, the endogenous TDA3 gene encoding Tda3p could be deleted in S. cerevisiae. Thus, the fungal cell preferably lacks a gene encoding Tda3p or comprises a disrupted endogenous gene encoding Tda3p.

In another embodiment, the modifications to the transport between the Golgi and the endosome involve disruption of Gos1p (SEQ ID NO: 2). This could be achieved by deleting the endogenous gene coding for this protein. For example, the endogenous GOS1 gene encoding Gos 1p could be deleted in S. cerevisiae.

Thus, another aspect of the embodiments relates to a fungal cell. According to the embodiments, the fungal cell lacks a gene encoding Gos1p or comprises a disrupted endogenous gene encoding Gos1p. The fungal cell also comprises a gene encoding a recombinant protein.

Hence, in an embodiment, the fungal cell lacks a gene encoding Gos1p or comprises a disrupted endogenous gene encoding Gos1p.

The two aspects described above can be combined. In such an approach, the fungal cells lacks a gene encoding Tda3p and lacks a gene encoding Gos1p; lacks a gene encoding Tda3p and comprises a disrupted endogenous gene encoding Gos1p; lacks a gene encoding Gos1p and comprises a disrupted endogenous gene encoding Tda3p; or comprises a disrupted endogenous gene encoding Tda3p and comprises a disrupted endogenous gene encoding Gos1p. The fungal cell also comprises a gene encoding a recombinant protein.

In an embodiment, any of the modifications above or below are combined with downregulation of proteins that form the retromer complex for transport from endosome to Golgi. This could be achieved by disrupting some of the proteins that make up this complex, such as Vps5p (SEQ ID NO:3), Vps17p (SEQ ID NO:4), Pep8p (SEQ ID NO:5), Vps29p (SEQ ID NO:6) and/or Vps35p (SEQ ID NO:7). For example, the endogenous genes VPS5, VPS17, PEP8, VPS29 and/or VPS35 that make up this complex could be deleted in S. cerevisiae.

Thus, in an embodiment, the fungal cell comprises a disrupted endogenous retromer complex for transport from endosome to Golgi.

In a particular embodiment, the fungal cell is genetically modified for reduced expression of at least one protein selected from a group consisting of Vps5p (SEQ ID NO:3), Vps17p (SEQ ID NO:4), Pep8p (SEQ ID NO:5), Vps29p (SEQ ID NO:6), Vps35p (SEQ ID NO:7), and variants thereof having at least 50% homology to any of SEQ ID NO:3-7).

In another embodiment, recombinant protein production in a fungal cell could be further increased by combining any modifications above or below with disruption in proteins that act as subunits of the HDA1 histone deacetylase complex. Deletion of such subunits increases protein production. Disruption of the HDA1 histone deacetylase complex could be achieved, for example, by disrupting the proteins that make up this complex, such as Hda2p (SEQ ID NO:8) and/or Hda3p (SEQ ID NO:9). This could be achieved by deleting the endogenous HDA2 and/or HDA3 genes encoding subunits of the HDA1 histone deacetylase complex in S. cerevisiae.

Thus, in an embodiment, the fungal cell lacks genes encoding subunits of HDA1 histone deacetylase complex, preferably at least one of Hda2p and Hda3p, or comprises disrupted endogenous genes encoding the subunits of HDA1 histone deacetylase complex, preferably at least one of Hda2p and Hda3p.

In another embodiment, recombinant protein production in a fungal cell could be increased by disruption of Pgm2p (SEQ ID NO: 10), encoding phosphoglucomutase, also referred to as phosphoglucomutase (alpha-D-glucose-1,6-bisphosphate-dependent) (EC 5.4.2.2). This can be achieved, for example, by deleting the endogenous gene encoding phosphoglucomutase. For example, the genes PGM2 and/or PGM1 could be deleted in S. cerevisiae.

Thus, in an embodiment, the fungal cell lacks genes encoding Pgm2p and/or Pgm1p or comprises a disrupted endogenous gene encoding Pgm2p and/or Pgm1p.

In another embodiment, recombinant protein production in a fungal cell could be increased by disruption of subunits of peroxisomal ABC transport complex. This can be achieved, for example, by deleting the endogenous genes encoding subunits of peroxisomal ABC transport complex, such as Pxalp (SEQ ID NO:11) and/or Pxa2p (SEQ ID NO:12). For example, the gene PXA1 and/or PXA2 could be deleted in S. cerevisiae.

Thus, in an embodiment, the fungal cell lacks genes encoding subunits of peroxisomal ABC transport complex, preferably at least one of Pxa1p and Pxa2p, or comprises disrupted endogenous genes encoding the subunits of peroxisomal ABC transport complex, preferably at least one of Pxa1P and Pxa2p.

In another embodiment, recombinant protein production in a fungal cell could be increased by disruption of members of the conserved endoplasmic reticulum membrane complex. For example, Emc1p (SEQ ID NO:13) could be disrupted. This can be achieved, for example, by deletion of the endogenous EMC1 gene in S. cerevisiae.

Thus, in an embodiment, the fungal cell lacks genes encoding members of the conserved endoplasmic reticulum membrane complex, preferably Emc1p, or comprises disrupted endogenous genes encoding the members of the conserved endoplasmic reticulum membrane complex, preferably Emc1p.

In another embodiment, recombinant protein production in a fungal cell could be increased by disruption of vesicle membrane receptor proteins, for example Snc1p (SEQ ID NO:14) and/or Snc2p (SEQ ID NO:15). This can be achieved, for example, by deletion of the endogenous SNC1 and/or SNC2 gene in S. cerevisiae.

Thus, in an embodiment, the fungal cell lacks genes encoding vesicle membrane receptor proteins, preferably at least one of Snc1p and Snc2p, or comprises disrupted endogenous genes encoding the vesicle membrane receptor proteins, preferably at least one of Snc1p and Snc2p.

In another embodiment recombinant protein production in a fungal cell could be increased by increasing the levels of proteins that act as components of the cytosolic tethering complex, such as Cog1p, Cog2p, Cog3p, Cog4p, Cog5p, Cog6p, Cog7p and/or Cog8p. This can be achieved through overexpression of the endogenous genes encoding these proteins. For example, in a preferred embodiment, the endogenous Cog5p (SEQ ID NO:16) protein is overexpressed in S. cerevisiae.

Thus, in an embodiment, the fungal cell is genetically modified for enhanced expression of at least one component of the cytosolic tethering complex, preferably the at least one component is selected from a group consisting of Cog1p, Cog2p, Cog3p, Cog4p, Cog5p, Cog6p, Cog7p and Cog8p.

In another embodiment, recombinant protein production in a fungal cell could be increased by combining any of the modifications described above or below with increases in the activities of chaperone proteins. This can be achieved by overexpression of proteins that act as chaperones. For example, the activity of the enzyme protein disulfide isomerase (PDI) (EC 5.3.4.1) could be increased by overexpression of the endogenous PDI1 gene (SEQ ID NO:17) in S. cerevisiae. This includes PDI yeast homologs, such as PDI1, MPD1, MPD2, EUG1, and EPS1. Alternatively, or in addition, other chaperones could be overexpressed. For example,

Binding immunoglobulin protein (BiP), encoded by KAR2 in S. cerevisiae, the thiol oxidase ERO1, encoded by ERO1, the Sm-like proteins SEC1 or SLY1, encoded by SEC1 and SLY1 could be overexpressed. Chaperones from other species could also, or alternatively, be introduced. For example, the mammalian co-chaperone GRP170 and the peptidyl-prolyl isomerase FKBP2 could be overexpressed in S. cerevisiae. Other genes that could be introduced into a fungal cell to further improve protein production include Dsbc and FkpA from Escherichia coli and S. cerevisiae peptidyl-prolyl cis-trans isomerase (encoded by CPR5).

In an embodiment, the fungal cell is genetically modified for enhanced expression of at least one endogenous chaperone protein, preferably the at least one endogenous protein is selected from a group consisting of Pd1p, Mpd1p, Mpd2p, Eug1p, Eps1p, Kar2p, Ero1p, Sec1p, Sly1p and Cpr5p.

In an embodiment, the fungal cell comprises at least one heterologous gene encoding a respective heterologous chaperone protein, preferably the respective heterologous chaperone is selected from a group consisting of mammalian GRP170, mammalian FKBP2, Escherichia coli Dsbc and E. coli FkpA.

In another embodiment, the activity of transcription factors that control the expression of protein chaperones could be increased in order to further increase recombinant protein production. For example, the activity of the transcription factor heat shock factor (HSF) could be increased by overexpression of the endogenous gene encoding HSF, such as HSF1 (SEQ ID NO:18) in S. cerevisiae. In addition, in another embodiment, a mutant version of HSF1 is expressed. For example, HSF1 from S. cerevisiae where arginine 206 is replaced, preferably by Serine (R206S), could be overexpressed in a fungal cell.

In an additional embodiment, the activity of the transcription factor Haclp (SEQ ID NO:19) is increased to activate the unfolded protein response to further facilitate protein folding and production. The activity of the transcription factor Hac1p could be increased by overexpression of the endogenous gene encoding Hac1p, such as HAC1 in S. cerevisiae.

Thus, in an embodiment, the fungal cell is genetically modified for enhanced expression of at least one transcription factor that controls the expression of chaperone proteins, preferably the at least one transcription factor is selected from a group consisting of Hsf1p and Hac1p.

In a further embodiment, the transport between the endoplasmic reticulum (ER) and the Golgi could be increased. This could be achieved, for example, by overexpression of the endogenous proteins involved in ER-Golgi transport, such as Ypt1p, Bos1p, Bet1p, Sec22p, Sed5p, Sar1p, Sec12p, Sec23p, Sec24p, Sec13p, sec14p, Sec15p, Sec16p, Sec17p, Sec18p, Sec19p, Sec20p, Sec21p, Sec22p, Sec25p, Sec26p, Sec27p, Sec28p, Sec29p, Sec30p, Sec31p, Erv14p, Erv26p, Emp24p, Erv25p and/or Erv29p. For example, any of the endogenous genes encoding for these activities, YPT1, BOS1, BET1, SEC22, SED5, SAR1, SEC12, SEC23, SEC24, SEC13, SEC14, SEC15, SEC16, SEC17, SEC18, SEC19, SEC20, SEC21, SEC22, SEC25, SEC26, SEC27, SEC28, SEC29, SEC30, SEC31, ERV14, ERV26, EMP24, ERV25 and ERV29, respectively, could be overexpressed in a S. cerevisiae cell. In a preferred embodiment the overexpressed gene is taken from the group of SEC12, SEC13, SEC16 and ERV25.

Thus, in an embodiment, the fungal cell is genetically modified for overexpression of at least one endogenous protein involved in the transport between the endoplasmic reticulum and the Golgi, preferably the at least one endogenous protein is selected from a group consisting of Ypt1p, Bos1p, Bet1p, Sec22p, Sed5p, Sar1p, Sec12p, Sec23p, Sec24p, Sec13p, sec14p, Sec15p, Sec16p, Sec17p, Sec18p, Sec19p, Sec20p, Sec21p, Sec22p, Sec25p, Sec26p, Sec27p, Sec28p, Sec29p, Sec30p, Sec31p, Erv14p, Erv26p, Emp24p, Erv25p and Erv29p.

In another embodiment recombinant protein production in a fungal cell is increased by combining any of the modifications described above or below with increase in the transport between the Golgi and the plasma membrane (PM). This can be achieved by increasing the levels of the vesicle components involved in Golgi-PM transport. For example, the levels of Sec3p, Sec5p, Sec10p, Sec6p, Sec8p, Exo70p, Exo84p, Sso1p, Sec1p, Ypt32p and/or Sec4p could be increased. This could be achieved by overexpression of any of the endogenous genes encoding these activities. For example, the endogenous genes SEC3, SEC5, SEC10, SEC6, SEC8, EXO70, EXO84, SSO1, SEC1, EXO70, YPT32 and/or SEC4 could be overexpressed in S. cerevisiae.

Thus, in an embodiment, the fungal cell is genetically modified for overexpression of at least one endogenous protein involved in the transport between the Golgi and the plasma membrane, preferably the at least one endogenous protein is selected from a group consisting of Sec3p, Sec5p, Sec10p, Sec6p, Sec8p, Exo70p, Exo84p, Sso1p, Sec1p, Ypt32p and Sec4p.

In some embodiments, the glycosylation of the fungal cell can be modified in order to achieve humanized glycosylation. This can be achieved, for example, by disruption of N-hypermannose glycosylation through disruption of Och1p, Alg3p and/or Mnn9p. For example, the endogenous genes encoding these proteins (OCH1, ALG3, and MNN9, respectively) could be deleted in S. cerevisiae.

In another embodiment, recombinant protein production in a fungal cell could be increased by combining any of the modifications described above or below with deletion in the lipid regulator Opi1p. For example, the endogenous gene encoding OPI1 could be deleted in S. cerevisiae.

In another embodiment, recombinant protein production in a fungal cell could be increased by combining any of the modifications described above or below with deletion of proteases in order to prevent proteolytic degradation of the target protein. This could include vacuolar proteases. For example, the vacuolar proteases Pep4p and/or Prb1p could be deleted. This could be achieve by deleting the endogenous genes PEP4 and/or PRB1 in S. cerevisiae. Alternatively, or in addition, yapsin proteases, which are a family of aspartic proteases located at cell surface could also be disrupted. For example, the yapsin proteases Yps1p, Yps2p, Yps3p, Yps5p, Yps6p and/or Yps7p could be downregulated. This could be achieved by deletion of the endogenous genes YPS1, YPS2, YPS3, YPS5, YPS6 and/or YPS7 in S. cerevisiae.

Thus, in an embodiment, the fungal cell lacks genes encoding proteases or comprises disrupted genes encoding endogenous proteases, preferably selected from a group consisting of Pep4p, Prb1p, Yps1p, Yps2p, Yps3p, Yps5p, Yps6p and Yps7p.

In a further embodiment, degradation of non-native proteins can be reduced by deletion or downregulation of the HTM1 gene in S. cerevisiae, coding for an alpha-1,2-specific exomannosidasc.

In another embodiment, recombinant protein production in a fungal cell could be increased by combining any of the modifications described above or below with increasing the levels of co-translational translocation components. This can be achieved, for example, by overexpression of the endogenous SRP components, such as Srp14p, Srp21p, Srp68p, Srp72p, Sec65p and/or Srp54p. For example, the endogenous genes SRP14, SRP21, SRP68, SRP72,SEC65 and/or SRP54 could be overexpressed in S. cerevisiae.

Thus, in an embodiment, the fungal cell is genetically modified for overexpression of at least one endogenous co-translational translocation protein, preferably selected from a group consisting of Srp14p, Srp21p, Srp68p, Srp72p, Sec65p and Srp54p.

In another embodiment, recombinant protein production in a fungal cell could be increased by combining any of the modifications described above or below with modifications in hypoxic gene expression. For example, the endogenous protein Rox1p could be disrupted by deletion of the ROX1 gene (a Heme-dependent repressor of hypoxic genes) in S. cerevisiae. Alternatively, activity of the transcription factor Upc2p could be increased. For example, the UPC2-1 allele, which has a G888D mutation in the C-terminus and as a result constitutively activates ergosterol biosynthesis could be overexpressed in S. cerevisiae.

In another embodiment, recombinant protein production in a fungal cell could be increased by combining any of the modifications described above or below with reduced endocytosis. This can be achieved by disrupting the endogenous proteins associated with endocytosis, such as Rvs161p and End3p. For example, the endogenous RVS161 and END3 genes could be downregulated in S. cerevisiae.

In another embodiment, recombinant protein production in a fungal cell could be increased by combining any of the modifications described above or below with disruption in vacuolar sorting. This could be achieved by disruption of genes involved in vacuolar sorting. For example, Vps30p, Rgp1p, Mrl1p, Vam3p, Vps2p, Vps3p, Vps4p, Vps11p, Vps13p, Vps16p, Vps18p, Vps20p, Vps22p, Vps23p, Vps24p, Vps25p, Vps27p, Vps28p, Vps31p, Vps32p, Vps33p, Vps36p, Vps37p, Vps39p, Vps41p, Vps43p, Vps44p and/or Vps46p.

Thus, in an embodiment, the fungal cell is genetically modified for downregulation of at least one protein involved in vacuolar sorting, preferably selected from a group consisting of Vps30p, Rgp1p, Mrl1p, Vam3p, Vps2p, Vps3p, Vps4p, Vps11p, Vps13p, Vps16p, Vps18p, Vps20p, Vps22p, Vps23p, Vps24p, Vps25p, Vps27p, Vps28p, Vps31p, Vps32p, Vps33p, Vps36p, Vps37p, Vps39p, Vps41p, Vps43p, Vps44p and Vps46p.

In an embodiment, the fungal cell lacks the gene encoding Tda3p or comprises a disrupted endogenous gene encoding Tda3p. The fungal cell is also genetically modified for reduced expression of Vps5p, such as lacks the gene encoding Vps5p or comprises a disrupted endogenous gene encoding Vps5p. The fungal cell further lacks a gene encoding Hda2p or comprises a disrupted endogenous gene encoding Hda2p.

In an embodiment, the fungal cell comprises a heterologous gene encoding the recombinant protein.

The above described embodiments may be combined.

Another aspect of the embodiments relates to a method for producing a recombinant protein. The method comprises culturing a fungal cell according to any of the embodiments in a culture medium and in culture conditions suitable for production of the recombinant protein by the fungal cell. The method also comprises collecting the recombinant protein from the culture medium and/or from the fungal cell.

### EXAMPLES

Example 1

### Effect of Single Deletions on Protein Production in Yeast

In this example the effect of single gene deletions of ECM3, EMC1, ERV29, GOS1, VPS5, TDA3, COG5, SNC2, HDA2, HDA3, TAN1, PGM2 and PXA1 on recombinant protein production and secretion was examined in a BY4742 S. cerevisiae strain.

These single gene deletion strains were purchased from the EUROSCARF and transformed with the α-amylase expression plasmid p426GPD-Amylase. Single gene deletion strains of BY4742 harboring plasmid p426GPD-Amylase were selected on SD-ura plates and then cultured in SD-2×SCAA medium for α-amylase production.

For protein production in tubes or shake flasks, yeast strains were cultured at 30° C. and 200 rpm for 96 hours in the SD-2×SCAA medium² containing 20 g/L glucose, 6.9 g/L yeast nitrogen base without amino acids, 190 mg/L Arg, 400 mg/L Asp, 1260 mg/L Glu, 130 mg/L Gly, 140 mg/L His, 290 mg/L Ile, 400 mg/L Leu, 440 mg/L Lys, 108 mg/L Met, 200 mg/L Phe, 220 mg/L Thr, 40 mg/L Trp, 52 mg/L Tyr, 380 mg/L Val, 1 g/L BSA, 5.4 g/L Na₂HPO₄, and 8.56 g/L NaH₂PO₄·H₂O (pH=6.0 by NaOH).

The α-amylase activity in culture supernatant was measured using the α-amylase assay kit (Megazyme K-CERA, Ireland) and a commercial α-amylase from Aspergillus oryzae (Sigma, USA) was used as a standard. The weight of α-amylase can be calculated with 69.6 U/mg as α-amylase conversion coefficient according to Liu et al (Biotechnol Bioeng. 2012 May; 109(5):1259-68. doi:10.1002/bit.24409). For intracellular α-amylase measurements, cell pellet was collected from 0.5 ml cell cultures by centrifugation at 12000×g for 3 min. The cell pellet was washed with distilled water and resuspended in 0.5 ml PBS buffer containing 5 μl halt protease inhibitor cocktail (Thermo Fisher, USA). The cell suspension was added to a lysing matrix tube and cell lysis was processed in a FastPrep-24 tissue and cell homogenizer (MP Biomedicals, USA) at a speed of 6.5 m/s for 2 min. Cell debris was removed by centrifugation and the supernatant fraction was used for α-amylase quantification.

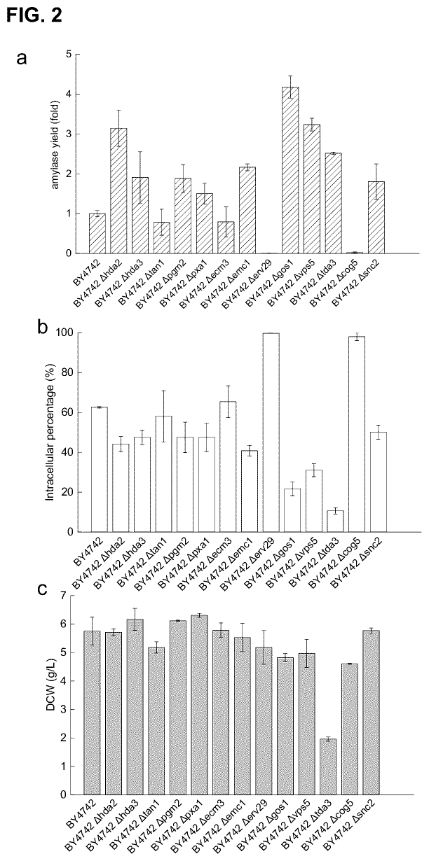

As shown in FIG. 2, amylase production was improved upon disruption of HDA2, HDA3, PGM2, PXA1, EMC1, GOS1, VPS5, TDA3 and SNC2 (FIG. 2a). Moreover, these modifications were generally associated with a decrease in the intracellular percentage of amylase (FIG. 2b), suggesting increased secretion.

### Example 2

### Combinatorial Effects of the Gene Deletions

The best four gene targets from Example 1 above (HDA2, VPS5, GOS1 and TDA3) were selected for further studies in CEN.PK strain background.

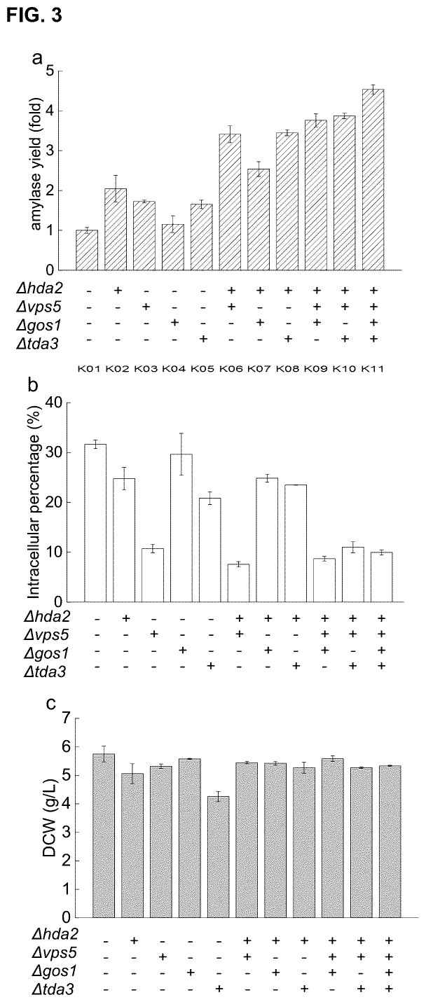

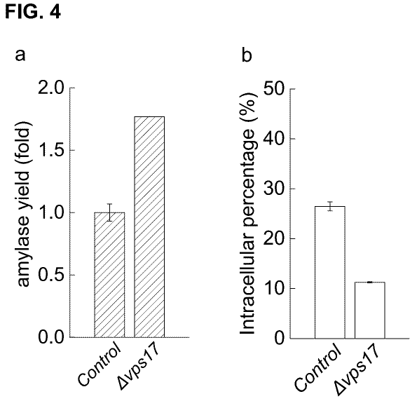

Gene deletion in CEN.PK strain was performed by using the amdS gene as selection marker and by following the protocols described by Solis-Escalante et al (FEMS Yeast Res. 2013 February; 13(1):126-39. doi:10.1111/1567-1364.12024). Primer pairs HDA2F and HDA2R and PrimeSTAR HS DNA polymerase (Takara, Kyoto, Japan) were used to amplify the HDA2 deletion cassette by using the plasmid pUG-amdSYM as template. The HDA2 deletion cassette was transformed into strain K01 for HDA2 deletion using a standard LiAc/SS DNA/PEG method by Gietz et al (Methods Enzymol. 2002 350, 87-96). Colonies grew on the selective SM-Ac plates were verified for correct hda2 deletion by diagnosis primers HDA2P1 and HDA2P2. As the primer HDA2R contains a homologous sequence to the upstream region of HDA2, the amdS marker can be looped out from the chromosome by homologous recombination. Similarly, VPS5, GOS1 and TDA3 deletion cassettes were amplified from the plasmid pUG-amdSYM by using primer pairs VPS5F/VPS5R, GOS1F/GOS1R and TDA3F/TDA3R, respectively. Deletion of VPS5, GOS1 and TDA3 in CEN.PK strain was carried out by transformation of deletion cassettes and selected on SM-Ac plates. Single gene deletion CEN.PK strains were cultured in SD-2×SCAA medium, and amylase secretion was measured as described in Example 1 above. As shown in FIG. 3, in all cases, single gene deletion increased amylase production and secretion. To further enhance amylase production, combinatorial gene deletions was performed. Combinatorial deletions further increased protein secretion, the triple gene deletions strain K10 (Δhda2, Δvps5 and Δtda3) can secrete 4 fold amylase compared with control strains in tube fermentation. It was noticed that deletion of VPS5 significantly reduced intracellular amylase retention, only 10% of amylase retained in strains with VPS5 deletion. As Vps5p formed a retromer subcomplex with Vps17p, we also tested deletion of VPS17 on amylase secretion. Deletion of VPS17 was performed by transformation of the VPS17 deletion cassette, which was amplified from the plasmid pUG-amdSYM by using primer pairs VPS17F/VPS17R. A similar result of amylase production was obtained in the VPS17 deletion strain, not only amylase yield increased, but also the retention of amylase significantly decreased (FIG. 4). This result emphasized the importance of trafficking between Golgi and endosome in protein secretion.

### Example 3

### Effect of ERV29 and COG5 Overexpression on Protein Production

The effect of overexpression of ERV29 and COG5 on protein secretion was also tested.

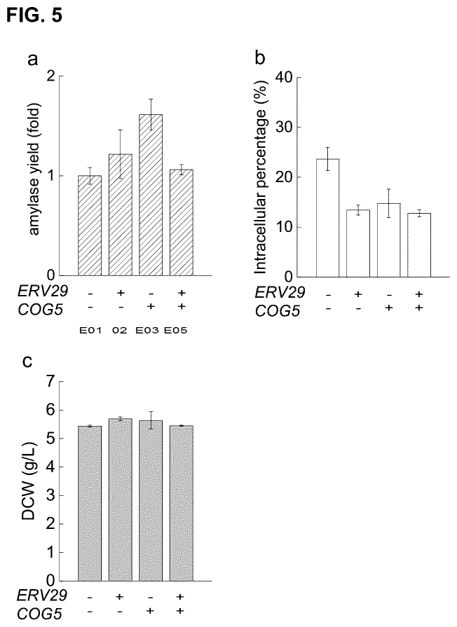

The ERV29 gene fragment was amplified from S. cerevisiae CEN.PK 530-1C genome by using primers ERV29EP1 and ERV29EP2, digested with restriction enzymes NotI and SacI, and inserted into the corresponding cloning sites of plasmid pSPGM1, resulting in plasmid pGM-ERV29. The ERV29 gene was under controlled by the promoter TEF1p on the plasmid pGM-ERV29. The COG5 gene fragment was amplified from S. cerevisiae CEN.PK 530-1C genome by using primers COG5EP1 and COGSEP2, digested with restriction enzymes BamHI and KpnI, and inserted into the corresponding cloning sites of plasmid pSPGM1, resulting in plasmid pGM-COG5. The COG5 gene was under controlled by the promoter PGK1p on the plasmid pGM-COG5. Similarly, the COG5 gene fragment was inserted into the BamHI-KpnI cloning sites of pGM-ERV29, resulting in the plasmid pGM-ERV-COG, which simultaneously overexpresses both ERV29 and COG5. Together with plasmid pAlphaAmyCPOT, plasmids pGM-ERV29, pGM-COG5 and pGM-ERV-COG were transformed to strain CEN.PK 530.1D as described in Example 1 above, resulting in strain E02, E03 and E05, respectively. Strain E01 with empty plasmid pSPGM1 was used as the reference strain. All strains were cultivated and analyzed for amylase production as described in Example 1 above. As shown in FIG. 5, single gene overexpression improved amylase secretion and decreased intracellular amylase retention. In contrast, combinatorial overexpression only decreased intracellular amylase retention but no increase in amylase secretion. The reason for no increase amylase secretion in combinatorial overexpression strain was most likely that overexpression of two genes by a high copy number plasmid increased burden of cells and consumed too much resource, which should be used for target protein.

### Example 4

### Combination of Gene Deletion and Gene Overexpression on Protein Production

To reduce cell burden and increase cell stability, strong promoter replacement was applied for overexpression of target genes. Promoter replacement was performed on the triple gene deletions strain K30 (deletion of HDA2, VPS5 and TDA3). The amdS-TEF1p cassette for replacement of the native ERV29 promoter was constructed as follows. Primers ERVPR1 and amdSR1 were used to amplify amdS marker by using plasmid pUG-amdSYM as template. Primers ERVPR3 and ERVPR4 were used to amplify the TEF1p fragment by using plasmid pGM-ERV29 as template. The amdS marker and TEFp fragment were fused together by fusion PCR and resulted in amdS-TEF1p cassette. The 5′ of amdS-TEF1p cassette is homologous to the upstream of the native ERV29 promoter and the 3′ of amdS-TEF1p cassette is homologous to the downstream of the native ERV29 promoter. Replacement of the native ERV29 promoter by the promoter TEF1p was accomplished by transformation of the amdS-TEF1p cassette to strain K30 and selected on SM-Ac plates. Primers ERV29P2 and ERVPR5 were used for verification of ERV29 promoter replacement. Similarly, the amdS-PGK1p cassette for replacement of the native COG5 promoter was constructed by using primer pairs COGPR1/amdSR1 and COGPR3/COGPR4, and plasmid pUG-amdSYM and pGM-COG5 as template, respectively. Replacement of the native COG5 promoter by the promoter PGK1p was accomplished by transformation of the amdS-PGK1p cassette to strain K30 and selected on SM-Ac plates, resulting in strain K13. Primers COG5P2 and COGPR5 were used for verification of COG5 promoter replacement.

We were also interested in whether overexpression of PDI1 is compatible with other gene target modifications and further increases protein production capacity of engineered strains. Therefore, both promoter replacement and gene integration were tested for the PDI1 gene. The native PDI1 promoter was replaced by a strong promoter FBA1p. The amdS-FBA1p cassette for replacement of the native PDI1 promoter was constructed as follows. Primers PDIFPR1 and amdSR1 were used to amplify amdS marker by using plasmid pUG-amdSYM as template. Primers PDIFPR3 and PDIFPR4 were used to amplify the FBAlp fragment by using S. cerevisiae CEN.PK 530-1C genome as template. The amdS marker and FBA1p fragment were fused together by fusion PCR and resulted in amdS-FBA1p cassette. As deletion of GOS1 showed positive on protein secretion, the position for integration of one copy of PDI1 gene was chosen in the GOS1 locus. Hence, integration of PDI1 was accomplished with replacement of GOS1. Two different PDI1 integration cassettes were tested. One was under controlled by the PDI1 native promoter PDI1p. Another one was under controlled by the promoter TEF1p. The amdS-PDI1p-PDI1 cassette for integration of PDI1 under control by the promoter PDI1p was constructed as follows. Primers GOSPDI1 and amdSR1 were used to amplified amdS marker by using plasmid pUG-amdSYM as template. Primers GOSPDI3 and GOSPDI4 were used to amplify the PDI1p-PDI1 fragment by using S. cerevisiae CEN.PK 530-1C genome as template. The amdS marker and PDI1p-PDI1 fragment were fused together by fusion PCR and resulted in amdS-PDI1p-PDI1 replacement cassette. The amdS-TEF1p-PDI1 cassette was constructed as follows. Primers NGOSPDI1 and amdSR1 were used to amplified amdS marker by using plasmid pUG-amdSYM as template. PDI1 gene fragment was amplified from S. cerevisiae CEN.PK 530-1C genome by using primers PDI1EP1 and PDIIEP2. The PDI1 gene fragment was then digested by NotI and SacI to insert after TEF1p on plasmid pSPGM1, resulting in pGM-PDI1. Primers NGOSPDI3 and NGOSPDI4 were used to amplify the TEF1p-PDI1 fragment by using pGM-PDI1 as template. The amdS marker and TEF1p-PDI1 fragment were fused together by fusion PCR and resulted in amdS-TEF1p-PDI1 replacement cassette. The amdS-FBA1p, amdS-PDI1p-PDI1 and amdS-TEF1p-PDI1 cassettes were transformed to yeast strain K40 for PDI1 promoter replacement or PDI1 integration, resulted in strain K15, K16 and K17, respectively. All transportations, cultivations and amylase measurements were carried out as described in Example1.

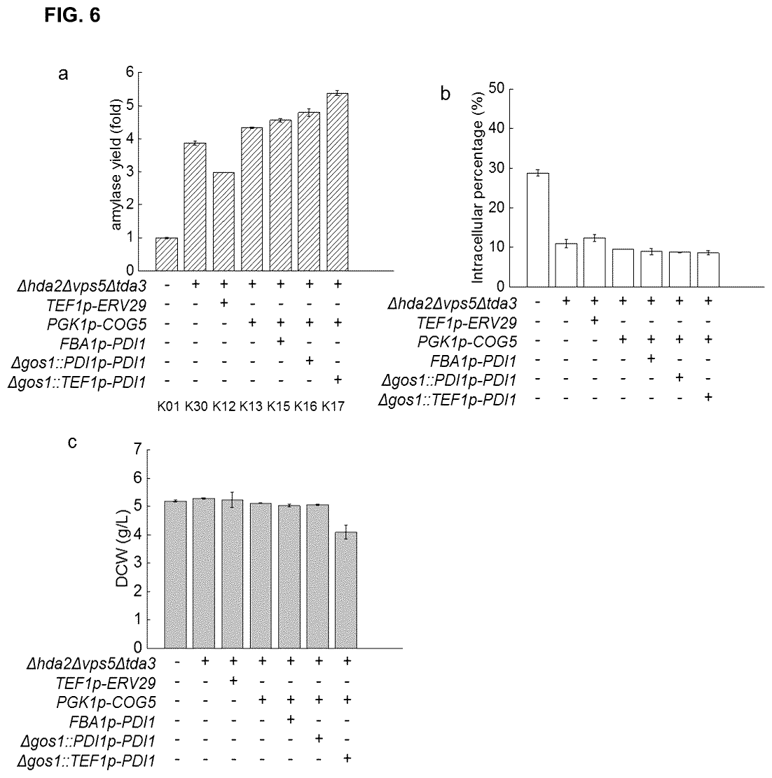

As shown in FIG. 6, overexpression of PDI1 was compatible with other gene modifications in yeast strains for enhancement of amylase secretion.

### Example 4

### Testing of Key Modifications with Other Proteins

In order to demonstrate that the modifications described herein are beneficial for different proteins, the best-producing strain was also tested with glucan 1,4-α-glucosidase instead of amylase. The amylase expression plasmid pAlphaAmyCPOT was eliminated from the best engineered strain K17 by serially transferring into non-selection YPE medium. Strain K17 without plasmid pAlphaAmyCPOT was renamed as CEN.PK 530-1CK303. Another plasmid pCP-aGLA, which expresses the glucan 1,4-α-glucosidase, was transformed into CEN.PK 530-1CK303, and colonies were selected on YPD plates. Then strain CEN.PK 530-1CK303 harboring plasmid pCP-aGLA was cultured in SD-2×SCAA medium and the glucan 1,4-α-glucosidase was measured by using Amyloglucosidase Assay Reagent (Megazyme, Ireland).

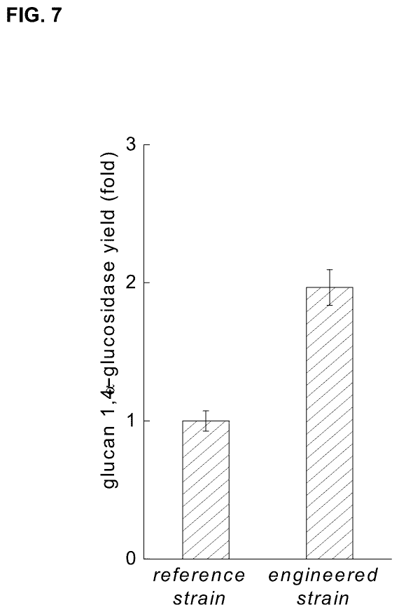

As shown in FIG. 7, compared with the reference strain, higher glucan 1,4-α-glucosidase yield was achieved by the engineered strain. This result supported that identified gene targets have a general positive effect on protein production, and can be widely used in construction of cell factories for protein production.

### Example 5

### Fed-Batch Fermentation and Fed-Batch Fermentation

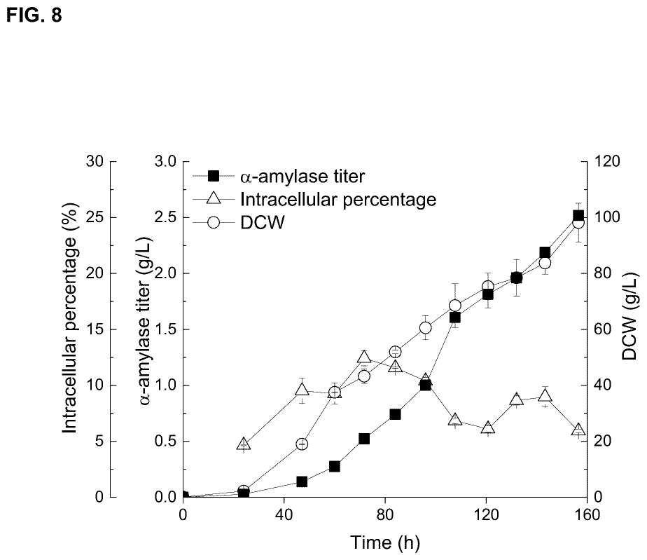

For the fed-batch cultivation, seed cultures of strain K17 was first inoculated to 200 ml SD-2×SCAA medium (5.4 g/L Na₂HPO₄ and 8.56 g/L NaH₂PO₄·H₂O were replaced by 2 g/L KH₂PO₄) with an initial OD₆₀₀ of 0.1. The bioreactor system was run at 30° C., 600 rpm as initial agitation speed and increased to maximally 1200 rpm, 18 L/h as initial air flow and increased to maximally 48 L/h. pH=6 (maintained by using 4 M KOH and 2 M HCl), the dissolved oxygen level was maintained above 30% by controlling agitation speed, air flow and medium feeding. Low glucose 10×feed medium contained: 200 g/L glucose, 69 g/L yeast nitrogen base without amino acids, 50 g/L casamino acids (Formedium, Norfolk, UK), 1 g/L BSA, 20 g/L KH2PO4 (pH=5 by KOH). For the high glucose 10×feed medium, 200 g/L glucose in low glucose 10×feed medium was replaced by 600 g/L glucose. After the glucose and ethanol were consumed in batch culture (200 ml SD-2×SCAA medium), the exponential feed was started by using the low glucose 10×feed medium and controlled at a specific growth rate of 0.08 h⁻¹. When both the agitation speed and the air flow reached maximum value (1200 rpm and 48 L/h, respectively), medium feeding was triggered by dissolved oxygen level >30%. After feeding about 330 ml of low glucose 10×feed medium, high glucose 10>feed medium was used. And fermentation was stopped when 330 ml of high glucose 10×feed medium was fed in the bioreactor. Totally, 660 ml of feeding medium was added to the bioreactor. Biological duplicate experiments were conducted in fed-batch cultivation. As shown in FIG. 8, the final α-amylase titer reached 2.5 g/L, and intracellular α-amylase retention maintained a low level (most of the time below 10% and peak value was 12%) in the whole process.

Both batch and fed-batch cultivation results confirmed that the protein secretion was substantially improved in yeast strain with combinatorial modifications. The engineered strain was able to adapt high density fermentation and showed potential industrial application.

TABLE 1
Plasmids and strains

| | | |
|---|---|---|
| Plasmids and strains | Relevant genotype | Reference |

Plasmids

| | | |
|---|---|---|
| CPOTud | 2 μm, AmpR, TPI1p, TPI1t, POT1 gene from | Biotechnol. Bioeng. |
| | S. pombe as a selection marker. | 109, 1259-1268 |
| | | (2012) |
| pAlphaAmyCPOT | CPOTud-(TPI1p-alpha factor | Biotechnol. Bioeng. |
| | leader-amylase gene-TPI1t) | 109, 1259-1268 |
| | | (2012) |
| p426GPD | 2 μm, AmpR, URA3, GPDp, CYC1t | Gene 156,119-122 |
| | | (1995). |
| p426GPD-Amylase | P426GPD-(GPDp-alpha factor leader-amylase | FEMS Yeast Res. |
| | gene-CYC1t) | 15, fov070 (2015). |
| pSPGM1 | 2 μm, AmpR, URA3, TEF1p, ADHI1t, PGK1p, CYC1t | FEMS Yeast Res. |
| | | 12, 598-607 |
| | | (2012). |
| pGM-ERV29 | pSPGM1-(TEF1p-ERV29-ADH1t) | This study |
| pGM-COG5 | pSPGM1-(PGK1p-COG5-CYC1t) | This study |
| pGM-ERV-COG | pSPGM1-(TEF1p-ERV29-ADH1t) + (PGK1p-COG5-CYC1t) | This study |
| pGM-PDI1 | pSPGM1-(TEF1p-PDI1-ADH1t) | This study |
| pCP-aGLA | CPOTud-(TPI1p-alpha factor leader-glucan | Proc. Natl Acad. |
| | 1,4-a-glucosidase gene-TPI1t) | Sci. USA 112, |
| | | E4689-E4696 |
| | | (2015). |
| pUG-amdSYM | AmpR, TEF2p-amdS-TEF2t | FEMS Yeast Res. |
| | | 13, 126-139 |
| | | (2013). |

Strains

| | | |
|---|---|---|
| BY4742 | MATα his3Δ1 leu2Δ0 lys2Δ0 ura3Δ0 | EUROSCARF |
| BY4742 Δhda2 | BY4742 Δhda2::kanMX4 | EUROSCARF |
| BY4742 Δhda3 | BY4742 Δhda3::kanMX4 | EUROSCARF |
| BY4742 Δtan1 | BY4742 Δtan1::kanMX4 | EUROSCARF |
| BY4742 Δpgm2 | BY4742 Δpgm2::kanMX4 | EUROSCARF |
| BY4742 Δpxa1 | BY4742 Δpxa1::kanMX4 | EUROSCARF |
| BY4742 Δecm3 | BY4742 Δecm3::kanMX4 | EUROSCARF |
| BY4742 Δemc1 | BY4742 Δemc1::kanMX4 | EUROSCARF |
| BY4742 Δerv29 | BY4742 Δerv29::kanMX4 | EUROSCARF |
| BY4742 Δgos1 | BY4742 Δgos1::kanMX4 | EUROSCARF |
| BY4742 Δvps5 | BY4742 Δvps5::kanMX4 | EUROSCARF |
| BY4742 Δtda3 | BY4742 Δtda3::kanMX4 | EUROSCARF |
| BY4742 Δcog5 | BY4742 Δcog5::kanMX4 | EUROSCARF |
| BY4742 Δsnc2 | BY4742 Δsnc2::kanMX4 | EUROSCARF |
| CEN.PK 530-1C | MATa URA3 HIS3 LEU2 TRP1 SUC2 MAL2-8^{c} | Biotechnol. Bioeng. |
| | tpi1(41-707)::loxP-kanMX-loxP | 109, 1259-1268 |
| | | (2012) |
| CEN.PK 530-1CK | MATa URA3 HIS3 LEU2 TRP1 SUC2 MAL2-8^{c} | This study |
| | tpi1(41-707)::loxP | |
| CEN.PK 530-1CK303 | CEN.PK 530-1CK Δhda2 Δvps5 Δtda3 PGK1p-COG5 | This study |
| | Δgos1::amdSYM-TEF1p-PDI1 | |
| CEN.PK 530-1D | MATa HIS3 LEU2 TRP1 SUC2 MAL2-8^{c} ura3-52 | Metab. Eng. 14, |
| | tpi1(41-707)::loxP-KanMX4-loxP | 120-127 (2012). |
| K01 | CEN.PK 530-1CK/pAlphaAmyCPOT | This study |
| K02 | CEN.PK 530-1CK Δhda2::amdSYM/ | This study |
| | pAlphaAmyCPOT | |
| K03 | CEN.PK 530-1CK Δvps5::amdSYM/ | This study |
| | pAlphaAmyCPOT | |
| K04 | CEN.PK 530-1CK Δgos1::amdSYM/ | This study |
| | pAlphaAmyCPOT | |
| K05 | CEN.PK 530-1CK Δtda3::amdSYM/ | This study |
| | pAlphaAmyCPOT | |
| K06 | CEN.PK 530-1CK Δhda2 Δvps5::amdSYM/ | This study |
| | pAlphaAmyCPOT | |
| K07 | CEN.PK 530-1CK Δhda2 Δgos1::amdSYM/ | This study |
| | pAlphaAmyCPOT | |
| K08 | CEN.PK 530-1CK Δhda2 Δtda3::amdSYM/ | This study |
| | pAlphaAmyCPOT | |
| K09 | CEN.PK 530-1CK Δhda2 Δvps5 Δgos1::amdSYM/ | This study |
| | pAlphaAmyCPOT | |
| K10 | CEN.PK 530-1CK Δhda2 Δvps5 Δtda3::amdSYM/ | This study |
| | pAlphaAmyCPOT | |
| K30 | CEN.PK 530-1CK Δhda2 Δvps5 Δtda3/ | This study |
| | pAlphaAmyCPOT | |
| K11 | CEN.PK 530-1CK Δhda2 Δvps5 Δtda3 | This study |
| | Δgos1::amdSYM/pAlphaAmyCPOT | |
| K12 | CEN.PK 530-1CK Δhda2 Δvps5 Δtda3 amdSYM- | This study |
| | TEF1p-ERV29/pAlphaAmyCPOT | |
| K13 | CEN.PK 530-1CK Δhda2 Δvps5 Δtda3 amdSYM- | This study |
| | PGK1p-COG5/pAlphaAmyCPOT | |
| K40 | CEN.PK 530-1CK Δhda2 Δvps5 Δtda3 PGK1p-COG5/ | This study |
| | pAlphaAmyCPOT | |
| K15 | CEN.PK 530-1CK Δhda2 Δvps5 Δtda3 PGK1p-COG5 | This study |
| | amdSYM-FBA1p-PDI1/pAlphaAmyCPOT | |
| K16 | CEN.PK 530-1CK Δhda2 Δvps5 Δtda3 PGK1p-COG5 | This study |
| | Δgos1::amdSYM-PDI1p-PDI1/pAlphaAmyCPOT | |
| K17 | CEN.PK 530-1CK Δhda2 Δvps5 Δtda3 PGK1p-COG5 | This study |
| | Δgos1::amdSYM-TEF1p-PDI1/pAlphaAmyCPOT | |
| E01 | CEN.PK 530-1D/pAlphaAmyCPOT + pSPGMI | This study |
| E02 | CEN.PK 530-1D/pAlphaAmyCPOT + pGM-ERV29 | This study |
| E03 | CEN.PK 530-1D/pAlphaAmyCPOT + pGM-COG5 | This study |
| E05 | CEN.PK 530-1D/pAlphaAmyCPOT + pGM-ERV-COG | This study |
| E13 | CEN.PK 530-1CK/pCP-aGLA | This study |
| E14 | CEN.PK 530-1CK303/pCP-aGLA | This study |

TABLE 2
Primers

| | | |
|---|---|---|
| Name | SEQ ID NO | Sequence (5′→3′)^{#} |

Plasmid construction

| | | |
|---|---|---|
| ERV29EP1 | 20 | ACTGCGGCCGCAACAAAATGTCTTACAGAGGACCTATTGGA |
| ERV29EP2 | 21 | CGTGAGCTCCTAGTAAATCTTCTTCTTTTCATCAACGGAT |
| COG5EP1 | 22 | TCAGGATCCAACAAAATGACAATAGCGCCAATGGCAA |
| COG5EP2 | 23 | CCAGGTACCTCACTTATTTAGAGAAATAGATACTGAGTTTAGCAT |
| PDI1EP1 | 24 | ACTGCGGCCGCAACAAAATGAAGTTTTCTGCTGGTGCC |
| PDI1EP2 | 25 | CGTGAGCTCTTACAATTCATCGTGAATGGCATCTTCT |

Gene deletion, gene replacement and promoter replacement

| | | |
|---|---|---|
| HDA2F | 26 | ATGAGTAGGAAAAATTCTAAGAAACTAAAAGTCTATTACTTACCT |
| | | GTAACGCTAACCCAAGACATGGAGGCCCAGA ATAC |
| HDA2R | 27 | AAATCTCTCTATATTATACAGGCTACTTCTTTTAGGAAACGTCAC |
| | | ATTCATTAGTCGATAGTATTGTATCTATTTTCTTTATTTTTCACAC |
| | | ACCAGTATAGCGACCAGCATTC |
| VPS5F | 28 | ATGGACTACGAGGATAATCTAGAAGCACCTGTTTGGGACGAACT |
| | | AAATCATGAGGGAGATAAAGACATGGAGGCCCAGA ATAC |
| VPS5R | 29 | ATAAATCCTGAGGAACGTGACACATAAAGTTATTGTATACAGAT |
| | | CATCTATTAGGCTTGTTATTGCAGGATGTATGAAAGTTTATAAAA |
| | | TCCCCAGTATAGCGACCAGCATTC |
| GOS1F | 30 | ATGAGCTCACAACCGTCTTTCGTCACCATAAGGGGCAAGGCCA |
| | | TTTCTCTAGAAACACAAACGGGACATGGAGGCCCAGA ATAC |
| GOS1R | 31 | AGATTCTTGTTATGTTTTTACATACGTTGTTTAATAAAAGTCGTTA |
| | | TTTATCAGTGGTGTGGTTGCTTGTCTGGAATTGGGCTTTTCCCTGT |
| | | GCAGTATAGCGACCAGCATTC |
| TDA3F | 32 | ATGGGTGAAGATTTTATGCACCCACCGTTTCAAACGTACCCTTC |
| | | AAAGAACAGCGAAGGGAAGACATGGAGGCCCAGA ATAC |
| TDA3R | 33 | CAAATTTGTGCATATACTTTTCTTGACCTTATTACTCCTCGGCTT |
| | | GATTATCATTATAAACACTATTCCTTCTGTTGCTTGGTTAAAATG |
| | | CTACAGTATAGCGACCAGCATTC |
| VPS17F | 34 | ATGACTTCGGCTGTACCTTATGATCCATATGATGATCTGGATAA |
| | | CAATCCATTTGCTGAGCCCCAGGAGGAAGACATGGAGGCCCAG |
| | | AATAC |
| VPS17R | 35 | AAAGATCACCTTGTTCAAAGGTATGAATTTTCTACTTTATATACG |
| | | TATTATCATGTTCAGAGGATAGATGGATTGACTAAGGGTACAGT |
| | | ACGGCAAACAGTATAGCGACCAGCATTC |
| amdSF1 | 36 | GACATG GAGGCCCAGA ATAC |
| amdSR1 | 37 | CAGTATAGCG ACCAGCATTC |
| ERVPR1 | 38 | TTCTAGAAGATGAGAGAAGAGGGAATAATGAGAAAGGCGAAAA |
| | | ATAAAGGCACACACCATAGCTTCAAAATGTTTCTACTCCTTTTTT |
| | | ACTCTTCCAGACATGGAGGCCCAGA ATAC |
| ERVPR3 | 39 | AAGTTAAGTGCGCAGAAAGTAATATCATGCGTCAATCGTATGTG |
| | | AATGCTGGTCGCTATACTGGCACACACCATAGCTTCAAAATGT |
| ERVPR4 | 40 | GACAAACTTGGAATGTAAGGCTTC |
| COGPR1 | 41 | ATTTTTTGTTAGACATATAATTTTATATCATTATTCTTATTATTCTT |
| | | ATAGGAAGTACCTTCAAAGAATGGGGTCTTATCTTGTTTTGCAA |
| | | GTACCACGACATGGAGGCCCAGA ATAC |
| COGPR3 | 42 | AAGTTAAGTGCGCAGAAAGTAATATCATGCGTCAATCGTATGTG |
| | | AATGCTGGTCGCTATACTGGGAAGTACCTTCAAAGAATGGGGTC |
| COGPR4 | 43 | TATCTCCAATGGGTTGCTATTCATC |
| PDIFPR1 | 44 | GCATTTTGTTGTGCTGTTACAACCACAACAAAACGAAAAACCCG |
| | | TATGGATCCAACTGGCACCGCTGGCTTGAACAACAATACCAGC |
| | | CTTCCAACTTCGACATGGAGGCCCAGA ATAC |
| PDIFPR3 | 45 | AAGTTAAGTGCGCAGAAAGTAATATCATGCGTCAATCGTATGTG |
| | | AATGCTGGTCGCTATACTGTCCAACTGGCACCGCTGGCTT |
| PDIFPR4 | 46 | ACAGCCTCTTGTTGGGCGAAAACAGAGGAGGCGAGCAGCAGG |
| | | GAGGACCATGACAGGACGGCACCAGCAGAAAACTTCATTTTGA |
| | | ATATGTATTACTTGGTTATGGTTATATATGAC |
| GOSPDI1 | 47 | GTTCAATAGTGTGGTTGGTAACCAAATTTTCTAGGCGTTGTTGA |
| | | AAATAATCATTAGTGCCCACCGTTTGAGCGTGGTGTGACACCAC |
| | | GCCCAAGATAGACATGGAGGCCCAGA ATAC |
| GOSPDI3 | 48 | CCCAGATGCGAAGTTAAGTGCGCAGAAAGTAATATCATGCGTCA |
| | | ATCGTATGTGAATGCTGGTCGCTATACTGTCATTAGTGCCCACC |
| | | GTTTGAG |
| GOSPDI4 | 49 | ATTACGAAATGGCCTGTATGGGTAGATTCTTGTTATGTTTTTACA |
| | | TACGTTGTTTAATAAAAGTCGTTATTCAATTACAATTCATCGTGA |
| | | ATGGCATCT |
| GOSPDI5 | 50 | CCCAGATGCGAAGTTAAGTGC |
| GOSPDI6 | 51 | ATTACGAAATGGCCTGTATGGGTAG |
| NGOSPDI1 | 52 | TACTCTTGTTCAATCAGTTAGTTATCTTTGTTCAATAGTGTGGTT |
| | | GGTAAGCACACACCATAGCTTCAAAATGTTTCTACTCCTTTTTTA |
| | | CTCTTCCAGACATGGAGGCCCAGA ATAC |
| NGOSPDI3 | 53 | CCCAGATGCGAAGTTAAGTGCGCAGAAAGTAATATCATGCGTCA |
| | | ATCGTATGTGAATGCTGGTCGCTATACTGGCACACACCATAGCT |
| | | TCAAAATGT |
| NGOSPDI4 | 54 | ATTACGAAATGGCCTGTATGGGTAGATTCTTGTTATGTTTTTACA |
| | | TACGTTGTTTAATAAAAGTCGTTATTCATTACAATTCATCGTGAA |
| | | TGGCATCTTC |

Verification primers for gene deletion, gene replacement and promoter
replacement

| | | |
|---|---|---|
| amdSP1 | 55 | TTACCACGGTGCTCCAGTTG |
| amdSP2 | 56 | AACCAAGTCAGCAGCAGAAG |
| HDA2P1 | 57 | TGCGGCACAGAAGAGTAACC |
| HDA2P2 | 58 | GGCGATAAACGATAGGCAAC |
| VPS5P1 | 59 | TCCGCTAAGAACAACTAAGTGA |
| VPS5P2 | 60 | CACTGGCTGTAAACGGACCTAT |
| GOS1P1 | 61 | TGCAAACCCAGTGTAAGACGC |
| GOS1P2 | 62 | ATATGGTTCGAGAACAGGCATC |
| TDA3P1 | 63 | AGCACGACATAGAAGTGAAACC |
| TDA3P2 | 64 | CGCAAGGGCAAACAGGATAGAC |
| VPS17P1 | 65 | CGATTGAGTCGAACACCCTGA |
| VPS17P2 | 66 | CTTGGGTGCGTAGGTCTGG |
| ERV29P2 | 67 | GTCTTGTAACCAATGGCGAAAC |
| ERVPR5 | 68 | GCCACCACGATTGACGAACA |
| COG5P2 | 69 | TTAACAGCGACTTGCCCACAGG |
| COGPR5 | 70 | AGCTAGTCTGTGACCTGTACG |
| PDIPR5 | 71 | TGCACGTGATAATATGTTACCCTGTC |
| PDIPR6 | 72 | GGAGGAGGATGAGATAAGTAGTTTCC |
| GOS1P5 | 73 | AAAACTCTGGCGGCTAAACTGG |
| GOS1P6 | 74 | CATCAATACTGGCGATAAGCGGGAC |
| PDI7 | 75 | TCCTTGGACTCTTTATTCGACTTCATC |
| PDI8 | 76 | CGCATTATAAGTGGTGTGCCGA |
| PDI10 | 77 | ATGCTGTGCTTGGGTGTTTTGA |

^{#}underlined sequence indicates restriction site.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1-12. (canceled)

13. A fungal cell, wherein said fungal cell is genetically modified for
(a) reduced expression of at least one protein selected from a group consisting of Pgm2, Pxa1p and Emc1, or
(b) overexpression of Cog1p, Cog2p, Cog3p, Cog4p, Cog5p, Cog6p, Cog7p or Cog8p, or any combination thereof; and
said fungal cell comprises a gene encoding a recombinant protein.

14. The fungal cell according to claim 13, wherein said fungal cell is genetically modified for enhanced expression of at least one endogenous chaperone protein.

15. The fungal cell according to claim 13, wherein said fungal cell is genetically modified for enhanced expression of at least one transcription factor that controls the expression of chaperone proteins, preferably said at least one transcription factor is selected from a group consisting of Hsf1p and Hac1p.

16. The fungal cell according to claim 13, wherein said fungal cell is genetically modified for overexpression of at least one endogenous protein involved in the transport between the endoplasmic reticulum and the Golgi, preferably said at least one endogenous protein is selected from a group consisting of Ypt1p, Bos1p, Bet1p, Sec22p, Sed5p, Sar1p, Sec12p, Sec23p, Sec24p, Sec13p, sec14p, Sec15p, Sec16p, Sec17p, Sec18p, Sec19p, Sec20p, Sec21p, Sec22p, Sec25p, Sec26p, Sec27p, Sec28p, Sec29p, Sec30p, Sec31p, Erv14p, Erv26p, Emp24p, Erv25p and Erv29p.

17. The fungal cell according to claim 13, wherein said fungal cell is genetically modified for overexpression of at least one endogenous protein involved in the transport between the Golgi and the plasma membrane, preferably said at least one endogenous protein is selected from a group consisting of Sec3p, Sec5p, Sec10p, Sec6p, Sec8p, Exo70p, Exo84p, Sso1p, Sec1p, Ypt32p and Sec4p.

18. The fungal cell according to claim 13, wherein said fungal cell lacks genes encoding proteases or comprises disrupted genes encoding endogenous proteases, preferably selected from a group consisting of Pep4p, Prb1p, Yps1p, Yps2p, Yps3p, Yps5p, Yps6p and Yps7p.

19. The fungal cell according to claim 13, wherein said fungal cell is genetically modified for downregulation of at least one protein involved in vacuolar sorting, preferably selected from a group consisting of Vps30p, Rgp1p, Mrl1p, Vam3p, Vps2p, Vps3p, Vps4p, Vps11p, Vps13p, Vps16p, Vps18p, Vps20p, Vps22p, Vps23p, Vps24p, Vps25p, Vps27p, Vps28p, Vps31p, Vps32p, Vps33p, Vps36p, Vps37p, Vps39p, Vps41p, Vps43p, Vps44p and Vps46p.

20. The fungal cell according to claim 13, wherein said fungal cell comprises a heterologous gene encoding said recombinant protein.

21. A method for producing a recombinant protein comprising:
culturing a fungal cell according to claim 13 in a culture medium and in culture conditions suitable for production of said recombinant protein by said fungal cell; and
collecting said recombinant protein from said culture medium and/or from said fungal cell.

22. The fungal cell according to claim 14, wherein said at least one endogenous chaperone protein is selected from a group consisting of Pd1p, Mpd1p, Mpd2p, Eug1p, Eps1p, Kar2p, Ero1p, Sec1p, Sly1p and Cpr5p
